SEQUENCE LISTING (10 sequences)

SEQ ID NO: 1 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - protein, 121 aa, Artificial
  Heavy chain variable domain
QVQLQESGPGLVKPSETLSLTCTVSGFSLTENSVSWIRQPPGKGLEWIGAVWSSGSTDYNSALKSRVTISRDTSKNQFSLKLSSVTAADTAVYYCTRSTYSNDFYYYFDYWGQGTMVTVSS

SEQ ID NO: 3 - protein, 108 aa, Artificial
  Light chain variable domain
SYELTQPPSVSVSPGQTASITCSGSELPKRYAYWYQQKPGQSIVRVIYKDSERPSGISERFSGSSSGNTATLTISGTQAMDEADYYCSSTYGDRKLPIFGGGTKLTVL

SEQ ID NO: 4 - protein, 13 aa, Artificial
  CDR3 of Heavy chain
STYSNDFYYYFDY

SEQ ID NO: 5 - protein, 16 aa, Artificial
  CDR2 of Heavy chain
AVWSSGSTDYNSALKS

SEQ ID NO: 6 - protein, 10 aa, Artificial
  CDR1 of Heavy chain
GFSLTENSVS

SEQ ID NO: 7 - protein, 11 aa, Artificial
  CDR3 of Light chain
SSTYGDRKLPI

SEQ ID NO: 8 - protein, 7 aa, Artificial
  CDR2 of Light chain
KDSERPS

SEQ ID NO: 9 - protein, 11 aa, Artificial
  CDR1 of Light chain
SGSELPKRYAY

SEQ ID NO: 10 - protein, 662 aa, Artificial
  Full-length antibody
QVQLQESGPGLVKPSETLSLTCTVSGFSLTENSVSWIRQPPGKGLEWIGAVWSSGSTDYNSALKSRVTISRDTSKNQFSLKLSSVTAADTAVYYCTRSTYSNDFYYYFDYWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSYELTQPPSVSVSPGQTASITCSGSELPKRYAYWYQQKPGQSIVRVIYKDSERPSGISERFSGSSSGNTATLTISGTQAMDEADYYCSSTYGDRKLPIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2022/0332827 A1
(43) Pub. Date: Oct. 20, 2022

(54) HUMANIZED ANTIBODIES AGAINST PD-L1

(71) Applicant: WUXI BIOLOGICS IRELAND LIMITED, CO LOUTH (IE)

(72) Inventors: Yong ZHENG, Shanghai (CN); Baotian YANG, Shanghai (CN); Jing LI, Shanghai (CN)

(21) Appl. No.: 17/636,874

(22) PCT Filed: Aug. 21, 2020
(86) PCT No.: PCT/CN2020/110494

§ 371 Date: Feb. 19, 2022

(30) Foreign Application Priority Data
Aug. 23, 2019 (CN) PCT/CN2019/102330

(51) Int. Cl. C07K 16/28; A61K 35/00

(52) U.S. Cl. CPC C07K 16/2827; A61K 35/00; C07K 2317/24; C07K 2317/33; C07K 2317/34; C07K 2317/76; C07K 2317/92

(57) ABSTRACT

Provided are humanized monoclonal antibodies against protein, programmed cell death ligand-1 (PD-L1), the methods of hybridoma generation using SD rats, the nucleic acid molecules encoding the anti-PD-L1 antibodies, expression vectors and host cells used for the expression of anti-PD-L1 antibodies. Also provided are the methods for validating the function of antibodies in vitro.

### TECHNICAL FIELD

The present invention relates generally to antibodies of PD-L1 and compositions thereof, and immunotherapy in the treatment of human disease using anti-PD-L 1 antibodies.

### BACKGROUND OF THE INVENTION

Increasing evidences from preclinical and clinical results have shown that targeting immune checkpoints is becoming the most promising approach to treat patients with cancers. The protein Programmed Death 1 (PD-1), an inhibitory member of the immunoglobulin super-family with homology to CD28, is expressed on activated B cells, T cells, and myeloid cells (Agata et al, supra; Okazaki et al (2002) Curr Opin. Immunol. 14: 391779-82; Bennett et al. (2003) J Immunol 170:711-8) and has a critical role in regulating stimulatory and inhibitory signals in the immune system (Okazaki, Taku et al. 2007 International Immunology 19:813-824). PD-1 was discovered through screening for differential expression in apoptotic cells (Ishida et al (1992) EMBO J 11: 3887-95).

The PD-1 is a type I transmembrane protein that is part of the Ig gene superfamily (Agata et al. (1996) bit Immunol 8:765-72) and the structure of PD-1 consists of one immunoglobulin variable-like extracellular domain and a cytoplasmic domain containing an immunoreceptor tyrosine-based inhibitory motif (ITIM) and an immunoreceptor tyrosine-based switch motif (ITSM). Although structurally similar to CTLA-4, PD-1 lacks the MYPPPY motif that is critical for B7-1 and B7-2 binding. PD-1 has two known ligands, PD-L1 (B7-H1, CD274) and PD-L2 (B7-DC, CD273), which are cell surface expressed members of the B7 family (Freeman et al (2000) J Exp Med 192: 1027-34; Latchman et al (2001) Nat Immunol 2:261-8; Carter etal (2002) Eur J Immunol 32:634-43). Both PD-L1 and PD-L2 are B7 homologs that bind to PD-1, but do not bind to other CD28 family members.

PD-1, as one of the immune-checkpoint proteins, is an inhibitory member of the CD28 family expressed on activated B cells, T cells, and myeloid cells (Agata etal, supra; Okazaki et al. (2002) Curr Opin Immunol 14: 391779-82; Bennett et al. (2003) J Immunol 170: 711-8) plays a major role in limiting the activity of T cells that provide a major immune resistance mechanism by which tumor cells escaped immune surveillance. PD-1 induces a state of anergy or unresponsiveness in T cells, resulting in the cells being unable to produce optimal levels of effector cytokines. PD-1 may also induce apoptosis in T cells via its ability to inhibit survival signals. PD-1 deficient animals develop various autoimmune phenotypes, including autoimmune cardiomyopathy and a lupus-like syndrome with arthritis and nephritis (Nishimura et al. (1999) Immunity 11:141-51; Nishimura et al. (2001) Science 291:319-22). Additionally, PD-1 has been found to play a role in autoimmune encephalomyelitis, systemic lupus erythematosus, graft-versus-host disease (GVHD), type I diabetes, and rheumatoid arthritis (Salama et al. (2003) J Exp Med 198:71-78: Prokunina and Alarcon-Riquelme (2004) Hum MoI Genet 13:R143; Nielsen et al. (2004) Lupus 11:510). In a murine B cell tumor line, the ITSM of PD-1 was shown to be essential to block BCR-mediated Ca²⁺-flux and tyrosine phosphorylation of downstream effector molecules (Okazaki etal. (2001) PNAS 98: 13866-71).

The interaction of PD-1 expressed on activated T cells, and PD-L1 expressed on tumor cells negatively regulates immune response and damps anti-tumor immunity. PD-L1 is abundant in a variety of human cancers (Dong et al (2002) Nat. Med 8:787-9). Expression of PD-L 1 on tumors is correlated with reduced survival in esophageal, pancreatic and other types of cancers, highlighting this pathway as a new promising target for tumor immunotherapy. Several groups have shown that the PD-1-PD-L interaction exacerbates disease, resulting in a decrease in tumor infiltrating lymphocytes, a decrease in T-cell receptor mediated proliferation, and immune evasion by the cancerous cells (Dong et al. (2003) J Mol. Med. 81:281-7; Blank et al. (2005) Cancer Immunol. Immunother. 54:307-314; Konishi et al. (2004) Clin. Cancer Res. 10:5094-100) Immune suppression can be reversed by inhibiting the local interaction of PD-1 with PD-L1, and the effect is additive when the interaction of PD-1 with PD-L2 is blocked as well.

Multiple agents targeting PD-1/PD-L 1pathway have been developed by big pharmaceutical companies, such as MedImmune, Bristol-Myers Squibb (BMS), Merck, Roche and GlaxoSmithKline (GSK). Data from clinical trials demonstrated early evidence of durable clinical activity and an encouraging safety profile in patients with various tumor types. Developed by MedImmune, Durvalumab is an engineered human monoclonal IgG1 antibody that acts against PD-L1, which grabbed the FDA's new breakthrough designation after impressive Phase I data came through for advanced metastatic urothelial bladder cancer. The results from a phase I study have shown an objective anti-lung cancer response in 39% of the PD-L1 positive patients and 5% of the PD-L1 negative patients. Nivolumab, an anti-PD-1 drug developed by BMS, is being put at center stage of the next-generation field. Now in 6 late-stage studies, the treatment spurred tumor shrinkage in three out of five cancer groups studied, including 18% of lung cancer patients (n=72), close to one third of melanoma patients (n=98) and 27% of patients with kidney cancer (n=33). Developed by Merck, Pembrolizumab is a humanized monoclonal IgG4 antibody that acts against PD-1, which grabbed the FDA's new breakthrough designation after impressive IB data came through for skin cancer. The results from a phase IB study have shown an objective anti-tumor response in 51% of the cancer patients (n=85), and a complete response in 9% of the patients.

There are some spaces for improvement for antibody against PD-L1as a therapeutic agent. Most of monoclonal antibodies against PD-L1currently tested in clinical trials are only against human PD-L1, which limits preclinical in vivo assay and diminished efficacy owing to the immunogenicity of the mouse-derived protein sequences. Humanized antibody with cross-reactivity to mouse PD-L1overcomes these shortages and showed more tolerability and higher efficiency in patients in vivo. Thus there is still a need for novel anti-PD-L1antibody.

### DISCLOSURE OF THE INVENTION

The present invention provides isolated antibodies, in particular monoclonal antibodies or human monoclonal antibodies.

In one aspect, the present invention provides an antibody or antigen binding fragment thereof human and murine PD-L 1 that binds to an epitope of PD-L 1 comprising amino acids at positions 19-22, 43-46, 48-49, 70, 118-120, 122 of SEQ ID NO:1.

The aforesaid antibody or the antigen binding fragment thereof, wherein the murine PD-L1is mouse or rat PD-L1.

The present invention provides an antibody or an antigen binding fragment thereof, comprising a heavy chain variable (VH) domain comprising an amino acid sequence that is at least 70%, 80%, 90% or 95% homologous to a sequence of SEQ ID NO: 2, wherein the antibody specifically binds to PD-L1.

The present invention provides an antibody or an antigen binding fragment thereof, further comprising a light chain variable (VL) domain comprising an amino acid sequence that is at least 70%, 80%, 90% or 95% homologous to a sequence of SEQ ID NO: 3.

The aforesaid antibody or the antigen binding fragment thereof, wherein the heavy chain variable (VH) domain comprises an amino acid sequence of SEQ ID NO: 2.

The aforesaid antibody or the antigen binding fragment thereof, wherein light chain variable (VL) domain comprises an amino acid sequence of SEQ ID NO: 3.

The present invention provides an antibody, or an antigen-binding fragment thereof, comprising:

a) a heavy chain variable (VH) domain comprising an amino acid sequence that is at least 70%, 80%, 90% or 95% homologous to a sequence of SEQ ID NOs: 2; and

b) a light chain variable (VL) domain comprising an amino acid sequence that is at least 70%, 80%, 90% or 95% homologous to a sequence of SEQ ID NOs: 3, wherein the antibody specifically binds to PD-L1.

The present invention provides an antibody or an antigen binding fragment thereof, comprising:
a) a heavy chain variable (VH) domain comprises an amino acid sequence of SEQ ID NOs: 2; and
b) light chain variable (VH) domain comprises an amino acid sequence of SEQ ID NOs: 3, wherein the antibody specifically binds to PD-L 1.

The present invention provides an antibody or an antigen binding fragment thereof, comprising: an amino acid sequence of SEQ ID NO:10.

The sequence of the said antibody is shown in Table 1 and Sequence Listing.

TABLE 1
Sequence of the antibody

| | | SEQ | |
|---|---|---|---|
| | | ID | |
| Clone ID | | NO | Amino acid sequence |
| W3152- | VH | 2 | QVQLQESGPGLVKPSETLSLTCTVSGFSLT |
| r11.135. | | | ENSVSWIRQPPGKGLEWIGAVWSSGSTDYN |
| 5-zAb17- | | | SALKSRVTISRDTSKNQFSLKLSSVTAADT |
| m6- | | | AVYYCTRSTYSNDFYYYFDYWGQGTMVTVS |
| uIgG4L | | | S |
| | VL | 3 | SYELTQPPSVSVSPGQTASITCSGSELPKR |
| | | | YAYWYQQKPGQSIVRVIYKDSERPSGISER |
| | | | FSGSSSGNTATLTISGTQAMDEADYYCSST |
| | | | YGDRKLPIEGGGTKLTVL |
| | Full | 10 | QVQLQESGPGLVKPSETLSLTCTVSGFSLT |
| | length | | ENSVSWIRQPPGKGLEWIGAVWSSGSTDYN |
| | | | SALKSRVTISRDTSKNQFSLKLSSVTAADT |
| | | | AVYYCTRSTYSNDFYYYFDYWGQGTMVTVS |
| | | | SASTKGPSVFPLAPCSRSTSESTAALGCLV |
| | | | KDYFPEPVTVSWNSGALTSGVHTFPAVLQS |
| | | | SGLYSLSSVVTVPSSSLGTKTYTCNVDHKP |
| | | | SNTKVDKRVESKYGPPCPPCPAPEFLGGPS |
| | | | VFLEPPKPKDTLMISRTPEVTCVVVDVSQE |
| | | | DPEVQFNWYVDGVEVHNAKTKPREEQFNST |
| | | | YRVVSVLTVLHQDWLNGKEYKCKVSNKGLP |
| | | | SSIEKTISKAKGQPREPQVYTLPPSQEEMT |
| | | | KNQVSLTCLVKGFYPSDIAVEWESNGQPEN |
| | | | NYKTTPPVLDSDGSFFLYSRLTVDKSRWQE |
| | | | GNVFSCSVMHEALHNHYTQKSLSLSLGKSY |
| | | | ELTQPPSVSVSPGQTASITCSGSELPKRYA |
| | | | YWYQQKPGQSIVRVIYKDSERPSGISERFS |
| | | | GSSSGNTATLTISGTQAMDEADYYCSSTYG |
| | | | DRKLPIFGGGTKLTVLGQPKAAPSVTLFPP |
| | | | SSEELQANKATLVCLISDFYPGAVTVAWKA |
| | | | DSSPVKAGVETTTPSKQSNNKYAASSYLSL |
| | | | TPEQWKSHRSYSCQVTHEGSTVEKTVAPTE |
| | | | CS |

In another aspect, the invention provides an antibody, or antigen-binding fragment thereof, comprising: a heavy chain variable region comprising CDR1, CDR2, and CDR3 sequences; and a light chain variable region comprising CDR1, CDR2, and CDR3 sequences, wherein the heavy chain variable region CDR3 sequence comprises a sequence of SEQ ID NO: 4, and conservative modifications thereof wherein the antibody specifically binds to PD-L 1.

Preferably, wherein the heavy chain variable region CDR2 sequence comprises a sequence of SEQ ID NO: 5, and conservative modifications thereof.

Preferably, the heavy chain variable region CDR1 sequence comprises a sequence of SEQ ID NO: 6, and conservative modifications thereof.

Preferably, the light chain variable region CDR3 sequence comprises a sequence of SEQ ID NO: 7, and conservative modifications thereof.

Preferably, the light chain variable region CDR2 sequence comprises a sequence of SEQ ID NO: 8, and conservative modifications thereof.

Preferably, the light chain variable region CDR1 sequence comprises a sequence of SEQ ID NO: 9, and conservative modifications thereof.

A preferred antibody comprises:
a) a heavy chain variable region CDR1 comprising SEQ ID NO: 6;
b) a heavy chain variable region CDR2 comprising SEQ ID NO: 5;
c) a heavy chain variable region CDR3 comprising SEQ ID NO: 4;
d) a light chain variable region CDR1 comprising SEQ ID NOs: 9;
e) a light chain variable region CDR2 comprising SEQ ID NOs: 8;
f) a light chain variable region CDR3 comprising SEQ ID NOs: 7;
wherein the antibody specifically binds to PD-L1.

The CDR sequence of the said antibody is shown in Table 2 and Sequence Listing.

TABLE 2
Sequence of the antibody

| | | SEQ | | SEQ | | SEQ | |
|---|---|---|---|---|---|---|---|
| | | ID | | ID | | ID | |
| Clone ID. | | NO | CDR1 | NO | CDR2 | NO | CDR3 |
| W3152- | Heavy | 6 | GFSLTENS | 5 | AVWSSGST | 4 | STYSNDFY |
| r11.135.5- | chain | | VS | | DYNSALKS | | YYFDY |
| zAb17-m6- | Light | 9 | SGSELPKR | 8 | KDSERPS | 7 | SSTYGDRK |
| uIgG4L | chain | | YAY | | | | LPI |

The antibodies of the invention can be humanized.

The antibodies of the invention can exhibit at least one of the following properties:
a) does not substantially bind to human PD-L2;
b) increases T-cell proliferation;
c) increases interferon-gamma production; or
d) increases interleukin-2 secretion.

In a further aspect, the invention provides a nucleic acid molecule encoding the antibody, or antigen binding fragment thereof.

The invention provides a cloning or expression vector comprising the nucleic acid molecule encoding the antibody, or antigen binding fragment thereof.

The invention also provides a host cell comprising one or more cloning or expression vectors.

In yet another aspect, the invention provides a process, comprising culturing the host cell of the invention and isolating the antibody, wherein the antibody is prepared through immunization in SD rat with human PD-L1extracellular domain and mouse PD-L1extracellular domain.

The invention provides a transgenic rat comprising human immunoglobulin heavy and light chain transgenes, wherein the mouse expresses the antibody of this invention.

The invention provides hybridoma prepared from the rat of this invention, wherein the hybridoma produces said antibody.

In a further aspect, the invention provides pharmaceutical composition comprising the antibody, or the antigen binding fragment of said antibody in the invention, and one or more of a pharmaceutically acceptable excipient, diluent or carrier.

The invention provides an immunoconjugate comprising the said antibody, or antigen-binding fragment thereof in this invention, linked to a therapeutic agent.

Wherein, the invention provides a pharmaceutical composition comprising the said immunoconjugate and a pharmaceutically acceptable excipient, diluent or carrier.

The invention also provides a method for preparing an anti-PD-L1antibody or an antigen-binding fragment thereof comprising:
(a) providing: (i) a heavy chain variable region antibody sequence comprising a CDR1 sequence of SEQ ID NO: 6, a CDR2 sequence of SEQ ID NO: 5; and a CDR3 sequence of SEQ ID NO: 4; and/or
(ii) a light chain variable region antibody sequence comprising a CDR1 sequence of SEQ ID NO: 9, a CDR2 sequence of SEQ ID NO: 8, and a CDR3 sequence of SEQ ID NO: 7; and
(b) expressing the altered antibody sequence as a protein.

The invention also provides a method of modulating an immune response in a subject comprising administering to the subject the antibody, or antigen binding fragment of any one of said antibodies in this invention.

The invention also provides the use of said antibody in the manufacture of a medicament for the treatment or prophylaxis of an immune disorder or cancer.

The invention also provides a method of inhibiting growth of tumor cells in a subject, comprising administering to the subject a therapeutically effective amount of the said antibody, or the said antigen-binding fragment to inhibit growth of the tumor cells.

Wherein, the invention provides the method, wherein the tumor cells are of a cancer selected from a group consisting of melanoma, renal cancer, prostate cancer, breast cancer, colon cancer, lung cancer, bone cancer, pancreatic cancer, skin cancer, cancer of the head or neck, cutaneous or intraocular malignant melanoma, uterine cancer, ovarian cancer, and rectal cancer.

Wherein, the invention provides the method, wherein the antibody is a chimeric antibody or humanized antibody.

The features and advantages in this invention

There are some spaces for improvement for antibody against PD-L1as a therapeutic agent. Most of monoclonal antibodies against PD-L1currently tested in clinical trials are only against human PD-L1, which limits preclinical in vivo assay and diminished efficacy owing to the immunogenicity of the mouse-derived protein sequences. Humanized antibody with cross-reactivity to mouse PD-L1overcomes these shortages and showed more tolerability and higher efficiency in patients. In this invention, we have generated a humanized antibody against PD-L1utilizing hybridoma technology. The antibodies reported in this invention have high binding affinity to both human and mouse PD-L1protein; with no cross-family reactions; potently block PD-L1 binding to CD80; and potently modulate immune responses, including enhancing T cell proliferation and increasing cytokine IFN-γ and interleukin-2 production; and have superior inhibitory efficacy against human Treg cells.

### BRIEF DESCRIPTION OF THE DRAWINGS

FIG. 1 shows the results of binding to cell surface human PD-L1by FACS FIG. 2 shows the results of binding to cell surface mouse PD-L1by FACS.

FIG. 3 shows the result of binding to cell surface cynomolgus PD-L1by FACS.

FIG. 4 shows show the results of cross-family binding test to cell surface human PD-L2.

FIG. 5 shows human PD-1/PD-L1 binding competition.

FIG. 6 shows mouse PD-1/PD-L1 binding competition.

FIG. 7 shows human PD-L1/CD80 binding competition.

FIG. 8 shows IL-2 secretion in human allogeneic MLR.

FIG. 9 shows IFN-γ secretion in human allogeneic MLR.

FIG. 10 shows T cell proliferation in human allogeneic MLR.

FIG. 11 shows IFN-γ secretion in human autologous MLR.

FIG. 12 shows T cell proliferation in human autologous MLR.

FIG. 13 shows the result of IFN-γ secretion of human CD4+T cells in the presence of Treg cells.

FIG. 14 shows T cell proliferation of human CD4+T cells in the presence of Treg cells.

FIG. 15 shows ADCC test of PD-L1antibody on dendritic cells FIG. 16 shows ADCC test of Herceptin on SK-Br-3.

FIG. 17 shows CDC test of PD-L1antibody on dendritic cells FIG. 18 shows CDC test of Rituximab on Ramos.

FIG. 19 shows epitope binning against WBP315BMK1.

FIG. 20 shows epitope binning against WBP315BMK6.

FIG. 21 shows binding to immobilized human PD-L1.

FIG. 22 shows hot spot residues of W3152-r11.135.5-zAb17-IgG4L mapped on human PD-L1structure.

FIG. 23 shows body weight of mice in control and treated groups.

FIG. 24 shows body weight change of mice in control and treated groups.

FIG. 25 shows tumor volume during treatment.

### DETAILED DESCRIPTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

The articles “a”, “an”, and “the” are used herein to refer to one or to more than one (i.e., to at least one) of the grammatical object of the article. By way of example, “a polypeptide complex” means one polypeptide complex or more than one polypeptide complex.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

Throughout this disclosure, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The terms “polypeptide”, “peptide”, and “protein” are used interchangeably herein to refer to a polymer of amino acid residues, or an assembly of multiple polymers of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine Amino acid analogs refer to compounds that have the same basic chemical structure as a naturally occurring amino acid, i.e., an alpha-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. An alpha-carbon refers to the first carbon atom that attaches to a functional group, such as a carbonyl. A beta-carbon refers to the second carbon atom linked to the alpha-carbon, and the system continues naming the carbons in alphabetical order with Greek letters Amino acid mimetics refers to chemical compounds that have a structure that is different from the general chemical structure of an amino acid, but that functions in a manner similar to a naturally occurring amino acid. The term “protein” typically refers to large polypeptides. The term “peptide” typically refers to short polypeptides. Polypeptide sequences are usually described as the left-hand end of a polypeptide sequence is the amino-terminus (N-terminus); the right-hand end of a polypeptide sequence is the carboxyl-terminus (C-terminus). “Polypeptide complex” as used herein refers to a complex comprising one or more polypeptides that are associated to perform certain functions. In certain embodiments, the polypeptides are immune-related.

The terms “Programmed Death 1”, “Programmed Cell Death 1”, “Protein PD-1”, “PD-1”, “PD1”, “PDCD1”, “hPD-1” and “hPD-F” are used interchangeably, and include variants, isoforms, species homologs of human PD-1, and analogs having at least one common epitope with PD-1.

The terms “Programmed Death ligand 1”, “PD ligand 1”, “PD-L1”, “PD L1”, “B7 homolog 1”, “B7-H1”, “B7 H1”, “CD274” are used interchangeably, and include variants, isoforms, species homologs of human PD-L1, and analogs having at least one common epitope with PD-L1.

The term “antibody” as referred to herein includes whole antibodies and any antigen-binding fragment (i.e., “antigen-binding portion”) or single chains thereof. An “antibody” refers to a protein comprising at least two heavy (H) chains and two light (L) chains inter-connected by disulphide bonds, or an antigen-binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The CDRs in heavy chain are abbreviated as H-CDRs, for example H-CDR1, H-CDR2, H-CDR3, and the CDRs in light chain are abbreviated as L-CDRs, for example L-CDR1, L-CDR2, L-CDR3.

The term “antibody” as used in this disclosure, refers to an immunoglobulin or a fragment or a derivative thereof, and encompasses any polypeptide comprising an antigen-binding site, regardless whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. The term “antibody” also includes antibody fragments such as scFv, dAb, bispecific antibodies comprising a first VH domain and a second VH domain, and other antibody fragments that retain antigen-binding function, i.e., the ability to bind PD-1 and LAG-3 specifically. Typically, such fragments would comprise an antigen-binding fragment.

An antigen-binding fragment typically comprises an antibody light chain variable region (VL) and an antibody heavy chain variable region (VH), however, it does not necessarily have to comprise both. For example, a so-called Fd antibody fragment consists only of a VH domain and CH1 domain, but still retains some antigen-binding function of the intact antibody.

The terms “antigen-binding fragment”, “antigen-binding domain”, and “binding fragment” refer to a part of an antibody molecule that comprises amino acids responsible for the specific binding between the antibody and the antigen. In instances, where an antigen is large, the antigen-binding fragment may only bind to a part of the antigen. A portion of the antigen molecule that is responsible for specific interactions with the antigen-binding fragment is referred to as “epitope” or “antigenic determinant”

An antigen-binding fragment typically comprises an antibody light chain variable region (VL) and an antibody heavy chain variable region (VH), however, it does not necessarily have to comprise both. For example, a so-called Fd antibody fragment consists only of a VH domain, but still retains some antigen-binding function of the intact antibody.

In line with the above the term “epitope” defines an antigenic determinant, which is specifically bound/identified by a binding fragment as defined above. The binding fragment may specifically bind to/interact with conformational or continuous epitopes, which are unique for the target structure, e.g. the human and murine PD-L1. A conformational or discontinuous epitope is characterized for polypeptide antigens by the presence of two or more discrete amino acid residues which are separated in the primary sequence, but come together on the surface of the molecule when the polypeptide folds into the native protein/antigen. The two or more discrete amino acid residues contributing to the epitope are present on separate sections of one or more polypeptide chain(s). These residues come together on the surface of the molecule when the polypeptide chain(s) fold(s) into a three-dimensional structure to constitute the epitope. In contrast, a continuous or linear epitope consists of two or more discrete amino acid residues, which are present in a single linear segment of a polypeptide chain.

The term “binds to an epitope of PD-L1” refers to the antibodies have specific binding for a particular epitope of PD-L 1, which may be defined by a linear amino acid sequence, or by a tertiary, i.e., three-dimensional, conformation on part of the PD-L1 polypeptide. Binding means that the antibodies affinity for the portion of PD-L1 is substantially greater than their affinity for other related polypeptides. The term “substantially greater affinity” means that there is a measurable increase in the affinity for the portion of PD-L1 as compared with the affinity for other related polypeptides. Preferably, the affinity is at least 1.5-fold, 2-fold, 5-fold 10-fold, 100-fold, 10³-fold, 10⁴-fold, 10⁵-fold, 10⁶-fold or greater for the particular portion of PD-L 1 than for other proteins. Preferably, the binding affinity is determined by enzyme-linked immunosorbent assay (ELISA), or by fluorescence-activated cell sorting (FACS) analysis or surface Plasmon resonance (SPR). More preferably, the binding specificity is obtained by fluorescence-activated cell sorting (FACS) analysis.

The term “cross-reactivity” refers to binding of an antigen fragment described herein to the same target molecule in human and murine (mouse or rat). Thus, “cross-reactivity” is to be understood as an interspecies reactivity to the same molecule X expressed in different species, but not to a molecule other than X. Cross-species specificity of a monoclonal antibody recognizing e.g. human PD-L 1, to a murine (mouse or rat) PD-L1, can be determined, for instance, by FACS analysis.

The terms “conservative modifications” i.e., nucleotide and amino acid sequence modifications which do not significantly affect or alter the binding characteristics of the antibody encoded by the nucleotide sequence or containing the amino acid sequence. Such conservative sequence modifications include nucleotide and amino acid substitutions, additions and deletions. Modifications can be introduced into the sequence by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

The term “homolog” and “homologous” as used herein are interchangeable and refer to nucleic acid sequences (or its complementary strand) or amino acid sequences that have sequence identity of at least 70% (e.g., at least 70%, 75%, 80%, 85%, 88%, 90%, 91%, 92%, 93%, 94%, 95%, 96%, 97%, 98%, 99%) to another sequences when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology

Information (NCBI), see also, Altschul S.F. et al., J. Mol. Biol., 215: 403-410 (1990); Stephen F. et al., Nucleic Acids Res., 25: 3389-3402 (1997)), ClustalW2 (available on the website of European Bioinformatics Institute, see also, Higgins D. G. et al., Methods in Enzymology, 266: 383-402 (1996); Larkin M.A. et al., Bioinformatics (Oxford, England), 23 (21): 2947-8 (2007)), and ALIGN or Megalign (DNAS TAR) software. Those skilled in the art may use the default parameters provided by the tool, or may customize the parameters as appropriate for the alignment, such as for example, by selecting a suitable algorithm

The term “specific binding” or “specifically binds” as used herein refers to a non-random binding reaction between two molecules, such as for example between an antibody and an antigen. In certain embodiments, the polypeptide complex and the bispecific polypeptide complex provided herein specifically bind an antigen with a binding affinity (K_{D}) of <10⁻⁶ M (e.g., <5 ×10⁻⁷ M, <2X 10⁻⁷ M, <10⁻⁷ M, <5 ×10⁻⁸ M, <2 ×10⁻⁸ M, <10 ⁻⁸ M, <5 ×10 ⁻⁹M, <2 ×10⁻⁹ M, <10⁻⁹ M, or <10 ⁻¹⁰ M). K_{D} as used herein refers to the ratio of the dissociation rate to the association rate (k off/k on), may be determined using surface plasmon resonance methods for example using instrument such as Biacore.

### Method of Preparation

The present disclosure provides isolated nucleic acids or polynucleotides that encode the polypeptide complex, and the bispecific polypeptide complex provided herein.

The term “nucleic acid” or “polynucleotide” as used herein refers to deoxyribonucleic acids (DNA) or ribonucleic acids (RNA) and polymers thereof in either single-or double-stranded form. Unless specifically limited, the term encompasses polynucleotides containing known analogues of natural nucleotides that have similar binding properties as the reference nucleic acid and are metabolized in a manner similar to naturally occurring nucleotides. Unless otherwise indicated, a particular polynucleotide sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19: 5081 (1991); Ohtsuka et al., J. Biol. Chem. 260: 2605-2608 (1985); and Rossolini et al., Mol. Cell. Probes 8: 91-98 (1994)) .

The nucleic acids or polynucleotides encoding the polypeptide complex and the bispecific polypeptide complex provided herein can be constructed using recombinant techniques. To this end, DNA encoding an antigen-binding moiety of a parent antibody (such as CDR or variable region) can be isolated and sequenced using conventional procedures (e.g., by using oligonucleotide probes that are capable of binding specifically to genes encoding the heavy and light chains of the antibody). Likewise, DNA encoding a TCR constant region can also be obtained. As an example, the polynucleotide sequence encoding the variable domain (VH) and the polynucleotide sequence encoding the first TCR constant region are obtained and operably linked to allow transcription and expression in a host cell to produce the first polypeptide. Similarly, polynucleotide sequence encoding VL are operably linked to polynucleotide sequence encoding second TCR constant region, so as to allow expression of the second polypeptide in the host cell. If needed, encoding polynucleotide sequences for one or more spacers are also operably linked to the other encoding sequences to allow expression of the desired product.

The encoding polynucleotide sequences can be further operably linked to one or more regulatory sequences, optionally in an expression vector, such that the expression or production of the first and the second polypeptides is feasible and under proper control.

The encoding polynucleotide sequence (s) can be inserted into a vector for further cloning (amplification of the DNA) or for expression, using recombinant techniques known in the art. In another embodiment, the polypeptide complex and the bispecific polypeptide complex provided herein may be produced by homologous recombination known in the art. Many vectors are available. The vector components generally include, but are not limited to, one or more of the following: a signal sequence, an origin of replication, one or more marker genes, an enhancer element, a promoter (e.g. SV40, CMV, EF-1α), and a transcription termination sequence.

The term “vector” as used herein refers to a vehicle into which a polynucleotide encoding a protein may be operably inserted so as to bring about the expression of that protein. Typically, the construct also includes appropriate regulatory sequences. For example, the polynucleotide molecule can include regulatory sequences located in the 5′-flanking region of the nucleotide sequence encoding the guide RNA and/or the nucleotide sequence encoding a site-directed modifying polypeptide, operably linked to the coding sequences in a manner capable of expressing the desired transcript/gene in a host cell. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating.

In some embodiments, the vector system includes mammalian, bacterial, yeast systems, etc., and comprises plasmids such as, but not limited to, pALTER, pBAD, pcDNA, pCal, pL, pET, pGEMEX, pGEX, pCI, pCMV, pEGFP, pEGFT, pSV2, pFUSE, pVITRO, pVIVO, pMAL, pMONO, pSELECT, pUNO, pDUO, Psg5L, pBABE, pWPXL, pBI, p 15TV-L, pPro18, pTD, pRS420, pLexA, pACT2.2 etc., and other laboratorial and commercially available vectors. Suitable vectors may include, plasmid, or viral vectors (e.g., replication defective retroviruses, adenoviruses and adeno-associated viruses).

Vectors comprising the polynucleotide sequence (s) provided herein can be introduced to a host cell for cloning or gene expression. The phrase “host cell” as used herein refers to a cell into which an exogenous polynucleotide and/or a vector has been introduced.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for the vectors encoding the polypeptide complex and the bispecific polypeptide complex. Saccharomyces cerevisiae, or common baker ‘ s yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16, 045), K. wickeramii (ATCC 24, 178), K. waltii (ATCC 56, 500), K.drosophilarum (ATCC 36, 906), K. thermotolerans, and K. marxianus; yarrowia (EP 402, 226); Pichia pastoris (EP 183, 070); Candida; Trichoderma reesia (EP 244, 234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated polypeptide complex, the bispecific polypeptide complex provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36: 59 (1977)), such as Expi293; baby hamster kidney cells (BHK, ATCC CCL 10); Chinese hamster ovary cells/-DHFR (CHO, Urlaub et al., Proc. Natl. Acad. Sci. USA 77: 4216 (1980)); mouse sertoli cells (TM4, Mather, Biol. Reprod. 23: 243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N. Y Acad. Sci. 383: 44-68 (1982)); MRC 5 cells; FS4 cells; and a human hepatoma line (Hep G2).

Host cells are transformed with the above-described expression or cloning vectors can be cultured in conventional nutrient media modified as appropriate for inducing promoters, selecting transformants, or amplifying the cloning vectors.

For production of the polypeptide complex and the bispecific polypeptide complex provided herein, the host cells transformed with the expression vector may be cultured in a variety of media. Commercially available media such as Ham’ s F10 (Sigma), Minimal Essential Medium (MEM), (Sigma), RPMI-1640 (Sigma), and Dulbecco ‘ s Modified Eagle’ s Medium (DMEM), Sigma) are suitable for culturing the host cells. In addition, any of the media described in Ham et al., Meth. Enz. 58: 44 (1979), Barnes et al., Anal. Biochem. 102: 255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN TM drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

In certain embodiments, the polypeptide complex or the bispecific polypeptide complex may be linked to a conjugate indirectly, or indirectly for example through another conjugate or through a linker. For example, the polypeptide complex or the bispecific polypeptide complex having a reactive residue such as cysteine may be linked to a thiol-reactive agent in which the reactive group is, for example, a maleimide, an iodoacetamide, a pyridyl disulphide, or other thiol-reactive conjugation partner (Haugland, 2003, Molecular Probes Handbook of Fluorescent Probes and Research Chemicals, Molecular Probes, Inc.; Brinkley, 1992, Bioconjugate Chem. 3: 2; Garman, 1997, Non-Radioactive Labelling: A Practical Approach, Academic Press, London; Means (1990) Bioconjugate Chem. 1: 2; Hermanson, G. in Bioconjugate Techniques (1996) Academic Press, San Diego, pp. 40-55, 643-671).

For another example, the polypeptide complex or the bispecific polypeptide complex may be conjugated to biotin, then indirectly conjugated to a second conjugate that is conjugated to avidin. For still another example, the polypeptide complex or the bispecific polypeptide complex may be linked to a linker which further links to the conjugate. Examples of linkers include bifunctional coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suherate), aldehydes (such as glutaraldehyde), bis-azido compounds (such as bis (p-azidobenzoyl) hexanediamine), bis-diazonium derivatives (such as bis- (p-diazoniumbenzoyl) -ethylenediamine), diisocyanates (such as toluene 2, 6-diisocyanate), and his-active fluorine compounds (such as 1, 5-difluoro-2, 4-dinitrobenzene) . Particularly preferred coupling agents include N-succinimidyl-3-(2-pyridyldithio) propionate (SPDP) (Carlsson et al., Biochem. J. 173: 723-737 (1978)) and N-succinimidyl-4-(2-pyridylthio) pentanoate (SPP) to provide for a disulphide linkage.

The conjugate can be a detectable label, a pharmacokinetic modifying moiety, a purification moiety, or a cytotoxic moiety. Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or (3-D-galactosidase), radioisotopes (e.g. 1231, 1241, 1251, 1311, 35S, 3H, 111In, 1121n, 14C, 64Cu, 67Cu, 86Y, 88Y, 90Y, 177Lu, 211At, 186Re, 188Re, 153Sm, 212Bi, and 32P, other lanthanides, luminescent labels), chromophoric moiety, digoxigenin, biotin/avidin, a DNA molecule or gold for detection. In certain embodiments, the conjugate can be a pharmacokinetic modifying moiety such as PEG which helps increase half-life of the antibody. Other suitable polymers include, such as, carboxymethylcellulose, dextran, polyvinyl alcohol, polyvinyl pyrrolidone, copolymers of ethylene glycol/propylene glycol, and the like. In certain embodiments, the conjugate can be a purification moiety such as a magnetic bead. A “cytotoxic moiety” can be any agent that is detrimental to cells or that can damage or kill cells. Examples of cytotoxic moiety include, without limitation, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Methods for the conjugation of conjugates to proteins such as antibodies, immunoglobulins or fragments thereof are found, for example, in U.S. Pat. Nos. 5,208,020; 6,441,163; WO2005037992; WO2005081711; and WO2006/034488, which are incorporated herein by reference to the entirety.

### Pharmaceutical Composition

The present disclosure also provides a pharmaceutical composition comprising the polypeptide complex or the bispecific polypeptide complex provided herein and a pharmaceutically acceptable carrier.

The term “pharmaceutically acceptable” indicates that the designated carrier, vehicle, diluent, excipient (s), and/or salt is generally chemically and/or physically compatible with the other ingredients comprising the formulation, and physiologically compatible with the recipient thereof.

A “pharmaceutically acceptable carrier” refers to an ingredient in a pharmaceutical formulation, other than an active ingredient, which is bioactivity acceptable and nontoxic to a subject. Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Method of treatment

Therapeutic methods are also provided, comprising: administering a therapeutically effective amount of the polypeptide complex or the bispecific polypeptide complex provided herein to a subject in need thereof, thereby treating or preventing a condition or a disorder. In certain embodiments, the subject has been identified as having a disorder or condition likely to respond to the polypeptide complex or the bispecific polypeptide complex provided herein.

As used herein, the term “subject” includes any human or nonhuman animal. The term “nonhuman animal” includes all vertebrates, e g, mammals and non-mammals, such as nonhuman primates, sheep, dogs, cats, horses, cows, chickens, amphibians, reptiles, etc. Except when noted, the terms “patient” or “subject” are used interchangeably.

The terms “treatment” and “therapeutic method” refer to both therapeutic treatment and prophylactic/preventative measures. Those in need of treatment may include individuals already having a particular medical disorder as well as those who may ultimately acquire the disorder.

In certain embodiments, the conditions and disorders include tumors and cancers, for example, non-small cell lung cancer, small cell lung cancer, renal cell cancer, colorectal cancer, ovarian cancer, breast cancer, pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphomas, myelomas, mycoses fungoids, merkel cell cancer, and other hematologic malignancies, such as classical Hodgkin lymphoma (CHL), primary mediastinal large B-cell lymphoma, T-cell/histiocyte-rich B-cell lymphoma, EBV-positive and -negative PTLD, and EBV-associated diffuse large B-cell lymphoma (DLBCL), plasmablastic lymphoma, extranodal NK/T-cell lymphoma, nasopharyngeal carcinoma, and HHV8-associated primary effusion lymphoma, Hodgkin's lymphoma, neoplasm of the central nervous system (CNS), such as primary CNS lymphoma, spinal axis tumor, brain stem glioma.

### Examples

### Example 1: Research materials preparation

1. Antigens and other proteins generation

Immunogen generation: DNAs encoding the extracellular domain (ECD) or full length of PD-1 and PD-L1 were synthesized and inserted into the expression vector pcDNA3.3. The inserted DNA sequences were verified by sequencing. Fusion proteins PD-1 ECD and PD-L1 ECD containing various tags, including human Fc, mouse Fc and His tags, were obtained by transfection of PD-1 or PD-L1 ECD gene into CHO or HEK293 cells. After 5 days, supernatants were harvested from the culture of transfected cells. The fusion proteins were purified and quantified for usage of immunization and screening.

The benchmark antibodies, namely WBP315BMK1 and WBP315BMK6, are applied as positive controls in the examples. These benchmark antibodies were constructed by fusing the variable domains with human IgG4 constant domain. The sequences of variable domain were from published patents. WBP315BMK1 was synthesized according to the clone of 12A4 from PCT publication No. WO2007005874 (BMS). WBP315BMK6 was synthesized according to the clone of 2.7A4 from U.S. patent No. U.S. Pat. No. 8,779,108 (Medimmune).

2. Cell lines establishment

In order to obtain tools for antibody screening and validation, PD-1 and PD-L 1 expressing cell lines were generated. Briefly, CHO-K1 or 293F cells were transfected with pcDNA3.3 expression vector containing full-length PD-1 or PD-L1 using Lipofectamine 2000 Transfection kit according to manufacturer's protocol. Two to three days post transfection, the transfected cells were cultured in medium containing Blasticidin or G418 to select the cells that had PD-1 or PD-L1 genes stably incorporated into their genomic DNAs. Meanwhile the cells were examined for the expression of the inserted genes. Once the expression was verified, single clones of interested were picked by limited dilution and scaled up to large volumes. The established monoclonal cell lines were then maintained in medium containing lower dose of antibiotics Blasticidin or G418.

### Example 2: Antibody hybridoma generation

1. Immunization

SD rats, at 6-8 weeks of age, were immunized with 10 μg/animal of human PD-L1 ECD protein and 10 μg/animal of mouse PD-L 1 ECD protein in TiterMax by footpad injection for prime, and were boosted twice a week with human PD-L1ECD protein or mouse PD-L 1 ECD protein in Aluminum alternately. The serum antibody titers were measured by Enzyme-Linked ImmunoSorbent Assay (ELISA) or flow cytometery (FACS) every two weeks.

2. Cell fusion

When the serum antibody titer was sufficiently high, rats were given a final boost with both human and mouse PD-L1ECD protein in D-PBS without adjuvant. The cell fusion was performed as follows: preparing myeloma cells SP2/0, myeloma cells were thawed one week before the fusion, and were split at 1:2 each day until the day before the fusion to keep in logarithmic growth. B lymphocytes isolated from lymph node of immunized SD rats were combined with myeloma cells (at 1:1 ratio). Cell mixture was washed and re-suspended in ECF solution at 2×10⁶ cells/mL for electronic cell fusion (ECF). Post ECF, cell suspension from the fusion chamber was immediately transferred into a sterile tube containing medium, and incubated for at least 24 hours in a 37° C. incubator before transferred into 96-well plates (1×10⁴ cells/well). The 96-well plates were cultured at 37° C., 5% CO₂, and were monitored periodically. When the clones were big enough, 100 tit of supernatant were transferred from the tissue culture plates to 96-well assay plates for screening.

3. First and confirmation screen of hybridoma supernatants

ELISA assay was used as first screening method to test the binding of hybridoma supernatants to human or mouse PD-L 1 protein. Briefly, plates (Nunc) were coated with either human or mouse PD-L1 ECD at 1 μg/mL overnight at 4° C. After blocking and washing, the hybridoma supernatants were loaded to the coated plates and incubated at room temperature for 1 hour. The plates were then washed and subsequently incubated with secondary antibody goat anti rat IgG HRP (Bethyl) for 45 min. After washing, TMB substrate was added and the reaction was stopped by 2M HCl. The absorbance at 450 nm was read using a microplate reader (Molecular Device).

In order to confirm the native binding of anti-PD-L 1 antibodies on conformational PD-L1 molecules expressed on cell membrane, FACS analysis was performed using PD-L1 transfected cell lines. CHO-K 1 cells expressing human PD-L1 or 293F cells expressing mouse PD-L1 were transferred into 96-well U-bottom plates (BD) at a density of 1×10⁵ cells/well. The hybridoma supernatants were then added and incubated with the cells for 1 hour at 4° C. After washing with 1 ><PBS/1% BSA, the secondary antibody goat anti rat FITC (Jackson ImmunoResearch Lab) was applied and incubated with cells at 4° C. in the dark for 1 hour. The cells were then washed and resuspended in 1 ><PBS/1% BSA or fixed with 4% paraformldehyde, and then analyzed by flow cytometery (BD). Antibody binding to parental CHO-K1 or 293F cell line was used as negative control, respectively.

To select potential antagonistic hits, selected antibodies were tested for their ability to block the binding of PD-1 to PD-L1 transfected cells by FACS analysis. CHO-K 1 cells expressing human PD-L 1 or 293F cells expressing mouse PD-L 1 were transferred into 96-well U-bottom plates (BD) at a density of 1×10⁵ cells/well. Hybridoma supernatants were added and incubated with the cells at 4° C. for 1 hour. After washing, mouse Fc fusion-human PD-1 protein or mouse Fc fusion-mouse PD-1 protein was added and incubated with cells at 4° C. for 1 hour. The secondary antibody goat anti mouse IgG Fc FITC antibody (no cross-reactivity to rat IgG Fc, Jackson ImmunoResearch Lab) was incubated with cells at 4° C. in the dark for 1 hour. The cells were then washed and resuspended in 1 ><PBS/1% BSA or fixed with 4% paraformaldehyde, and then analyzed by flow cytometry (BD).

4. Hybridoma subcloning

Once specific binding and blocking activity were verified through first and confirmation screening, the positive hybridoma cell lines were used for subcloning. Briefly, for each hybridoma cell line, cells were counted and diluted to give 5 cells, 1 cell or 0.5 cell per 200 μL cloning medium. The cell suspension was plated 200 μL/well into 96-well plates, one plate at 5 cells/well, one plate at 1 cell/well and four plates at 0.5 cell/well. Plates were cultured at 37° C., 5% CO₂, till they were ready to be screened by binding ELISA as described above. The exhausted supernatant of selected single clones were collected, and the antibodies were purified for further characterization.

### Example 3: Antibody hybridoma cell sequence and humanized antibody molecules construction and affinity maturation 1. Antibody hybridoma cell sequence

RNAs were isolated from monoclonal hybridoma cells with Trizol reagent. The VH and VL of anti-PD-L1chimeric antibodies were amplified as follows: RNA is first reverse transcribed into cDNA using a reverse transcriptase as described here, Reaction system (20 μL)

| | |
|---|---|
| 10 × RT Buffer | 2.0 μL |
| 25 × dNTP Mix (100 mM) | 0.8 μL |
| 10 × RT Random Primers/oligo dT/specific primer | 2.0 μL |
| MultiScribe ™ Reverse Transcriptase | 1.0 μL |
| RNase Inhibitor | 1.0 μL |
| RNA | 2 μg |
| Nuclease-free H₂O | to 20.0 μL |

Reaction condition

| | Step 1 | Step 2 | Step 3 | Step 4 |
|---|---|---|---|---|
| Temperature | 25° C. | 37° C. | 85° C. | 4° C. |
| Time | 10 min | 120 min | 5 min | ∞ |

The resulting cDNA was used as templates for subsequent PCR amplification using primers specific for interested genes. The PCR reaction was done as follows:

| | |
|---|---|
| cDNA | 1.0 μL |
| Ex PCR buffer | 5.0 μL |
| dNTP | 2.0 μL |
| ExTaq | 0.5 μL |
| P1 (25 pM) | 0.5 μL |
| P2 (25 pM) | 0.5 μL |
| ddH₂O | 40.5 μL |

Reaction condition:

| 94° C. | 3 min | | |
|---|---|---|---|
| 94° C. | 30 s | | |
| | | {close oversize brace} | 30 cycles |

| | |
|---|---|
| 60° C. | 30 s |
| 72° C. | 1 min |
| 72° C. | 10 min |

The resulting PCR product (10 μL) was ligated with pMD18-T vector. Top 10 competent cells were transformed with 10 μL of the ligation product. Positive clones were checked by PCR using M13-48 and M13-47 primers followed by sequencing.

2. Humanized antibody molecule construction

“Best Fit” approach was used to humanize anti-PD-L1antibody's light and heavy chains. For light chains amino acid sequences of corresponding V-genes were blasted against in-house human germline V-gene database. The sequence of humanized VL-gene was derived by replacing human complementary-determining region (CDR) sequences in the top two hits with rat CDR sequences using Kabat CDR definition. For heavy chains humanized sequences were derived by blasting rat frameworks against human germline V-gene database. Frameworks were defined using extended CDR definition where Kabat CDR1 was extended by five amino acids at N-terminus. Top two hits were used to derive sequences of humanized VH-genes. Humanized genes were back-translated, codon-optimized for mammalian expression, and synthesized by GeneArt Costum Gene Synthesis (Life Technologies), to express humanized antibodies.

Humanization by CDR grafting may result in partial or complete loss of binding. In order to restore the binding, human-to-mouse back mutations were introduced into humanized VH and VL genes by side directed mutagenesis. Specific amino acid substitutions were selected based on structural modeling of original rat and humanized antibodies and using information from previous humanization projects. Four humanized VH genes containing three back mutations in frameworks two and three, and five humanized VL genes containing four back mutations in frameworks two and three. All mutations were introduced using Quick Change site-directed mutagenesis kit (Agilent Technologies).

Hybridoma clone W3152-r11.135.5 was selected for humanization, the chimeric antibody of which was named W3152-r11.135.5.xAb.IgG4L (W3152-r11.135.5.xAb.IgG4X). Table 3 shows the result of expressed supernatants of humanized candidates binding to cell surface human PD-L1.

TABLE 3
Humanized variants binding to cell surface human PD-Ll

| | Binding to Cell Surface | |
|---|---|---|
| | human PD-Ll (MFI) | |
| Antibody | 1:1* | 1:10* |
| W3152-r11.135.5.xAb.IgG4L | 2417 | 2062 |
| W3152-r11.135.5-zAb1-IgG4L | 22.6 | 24.7 |
| W3152-r11.135.5-zAb2-IgG4L | 17.9 | 22.6 |
| W3152-r11.135.5-zAb3-IgG4L | 17.8 | 20.4 |
| W3152-r11.135.5-zAb4-IgG4L | 17.3 | 25.2 |
| W3152-r11.135.5-zAb5-IgG4L | 27.6 | 29.3 |
| W3152-r11.135.5-zAb6-IgG4L | 26.4 | 29.2 |
| W3152-r11.135.5-zAb7-IgG4L | 28.4 | 27 |
| W3152-r11.135.5-zAb8-IgG4L | 647 | 203 |
| W3152-r11.135.5-zAb9-IgG4L | 27.5 | 28.5 |
| W3152-r11.135.5-zAb10-IgG4L | 798 | 275 |
| W3152-r11.135.5-zAb11-IgG4L | 1552 | 722 |
| W3152-r11.135.5-zAb12-IgG4L | 2471 | 2059 |
| W3152-r11.135.5-zAb13-IgG4L | 1570 | 699 |
| W3152-r11.135.5-zAb14-IgG4L | 2461 | 2023 |
| W3152-r11.135.5-zAb15-IgG4L | 1654 | 845 |
| W3152-r11.135.5-zAb16-IgG4L | 2429 | 1979 |
| W3152-r11.135.5-zAb17-IgG4L | 2406 | 1998 |

3. Affinity maturation

Each amino acid of five CDRs (VHCDR1, VHCDR2, VHCDR3, VLCDR1 and VLCDR3) of parental clone W3152-r11.135.5-zAb17-IgG4L was individually mutated to other 20 amino acids using hybridization mutagenesis method. DNA primers containing a NNS codon encoding twenty amino acids were used to introduce mutation to each targeted CDR position. The individual degenerate primers were used in hybridization mutagenesis reactions. Briefly, each degenerate primer was phosphorylated, and then used in a 10:1 ration with uridylated ssDNA. The mixture was heated to 85° C. for 5 minutes then cooled down to 55° C. over 1 hours. Thereafter, T4 ligase and T4 DNA polymerase were added and mix was incubated for 1.5 hours at 37° C. Synthesis products for VH and VL CDRs were pooled respectively. Typically, 200 ng of the pooled library DNA was electroporated into BL21 for plaque formation on BL21 bacterial lawn or for production of scFv fragments.

The primary screening consisted of s single point ELISA (SPE) assay which was carried out using periplasmic extract (PE) of bacteria grown in 96-well deep-well plates. Briefly, this capture ELISA involved coating individual wells of a 96-well Maxisorp Immunoplate with anti-c-myc antibody in coating buffer (200 mM Na₂CO₃/NaHCO₃) at pH 9.2 overnight at 4° C. The next day, the plate was blocked with Casein for 1 hour at ambient temperature. scFv PE was then added to the plate and incubated at ambient temperature for 1 hour. After washing, biotinylated antigen protein was added to the well and the mixture was incubated for 1 hour at ambient temperature. This was followed by incubation with HRP conjugated for 1 hour at ambient temperature. HRP activity was detected with TMB substrate and the reaction quenched with 2M HCl. Plates were read at 450 nm. Clones exhibiting an optical density (OD) signal at 450 nm greater than the parental clone were picked and re-assayed by ELISA (as described above) in duplicate to confirm positive results. Clones that repeatedly exhibited a signal greater than that of the parental antibody were sequenced. The scFv protein concentration of each clone that had a CDR change was then determined by a quantitative scFv ELISA, where a scFv with known concentration was used as a reference. The scFv protein concentration was determined by comparing the ELISA signals with signals generated by the reference scFv. The binding assay was repeated once more for all positive variants under normalized scFv concentration in order to determine the relative binding affinity of the mutated scFv and the parental antibody.

The point mutations in VH and VL determined to be beneficial for binding to antigen were further combined to gain additional binding synergy. The combinatorial mutants were expressed as scFv and screened using the capture ELISA. Clones exhibiting an OD signal at 450 nm greater than the parental clone were sequenced and further ranked by binding ELISA as described above.

After affinity maturation, a total of 17 top-ranked clones (m1-m17) were obtained. Table 4 shows the results of affinity data of these 17 clones to human, cynomolgus monkey and mouse PD-L1. The 17 clones were re-formatted on human IgG4 backbone and further confirmed by SPR. The Koff data of these candidates to human PD-L 1 is shown in Table 5. W3152-r11.135.5-zAb17-m6-uIgG4L, also named “315E”, was selected for further characterization.

TABLE 4
Affinity to human, cynomolgus monkey and mouse PD-L1 by ELISA

| | Kd | Bmax | Kd | Bmax | Kd | Bmax | Competition |
|---|---|---|---|---|---|---|---|
| Name | (human) | (human) | (cyno) | (cyno) | (mouse) | (mouse) | capture ELISA |
| W315-BMK1-scFv | 0.000993 | 3.372 | 0.03730 | 1.305 | N/A | 0.08281 | 3.1435 |
| W3152-r11.135.5-zAb17-scFV | 0.3319 | 1.957 | N/A | 0.06424 | N/A | 0.07287 | 0.1226 |
| W3152-r11.135.5-zAb17-m1 | 0.000784 | 3.229 | 0.007496 | 2.434 | 0.01024 | 3.224 | 3.08925 |
| W3152-r11.135.5-zAb17-m2 | 0.000812 | 3.25 | 0.006295 | 2.243 | 0.317 | 3.38 | 3.0774 |
| W3152-r11.135.5-zAb17-m3 | 0.000755 | 3.199 | 0.006248 | 2.434 | 0.01811 | 3.365 | 3.13305 |
| W3152-r11.135.5-zAb17-m4 | 0.000802 | 3.238 | 0.005941 | 2.255 | 0.3316 | 3.995 | 2.93745 |
| W3152-r11.135.5-zAb17-m5 | 0.000908 | 3.183 | 0.009514 | 2.337 | 0.01493 | 3.38 | 3.104 |
| W3152-r11.135.5-zAb17-m6 | 0.000671 | 3.221 | 0.005089 | 2.403 | 0.01604 | 3.456 | 3.17125 |
| W3152-r11.135.5-zAb17-m7 | 0.001004 | 3.248 | 0.01064 | 2.127 | 0.0259 | 3.58 | 3.1334 |
| W3152-r11.135.5-zAb17-m8 | 0.001226 | 3.24 | 0.008089 | 1.934 | 0.3175 | 3.682 | 3.003 |
| W3152-r11.135.5-zAb17-m9 | 0.001018 | 3.253 | 0.006812 | 2.16 | 0.01181 | 3.431 | 3.02105 |
| W3152-r11.135.5-zAb17-m10 | 0.000705 | 3.193 | 0.006097 | 2.391 | 0.01024 | 3.224 | 3.0596 |
| W3152-r11.135.5-zAb17-m11 | 0.000872 | 3.209 | 0.008051 | 2.227 | 0.09144 | 3.469 | 3.1002 |
| W3152-r11.135.5-zAb17-m12 | 0.000587 | 3.184 | 0.004988 | 2.299 | 0.317 | 3.38 | 3.0609 |
| W3152-r11.135.5-zAb17-m13 | 0.000931 | 3.267 | 0.005992 | 2.439 | 0.01811 | 3.365 | 3.02545 |
| W3152-r11.135.5-zAb17-m14 | 0.000691 | 3.178 | 0.004507 | 2.461 | 0.3316 | 3.995 | 2.8014 |
| W3152-r11.135.5-zAb17-m15 | 0.000381 | 3.188 | 0.002638 | 2.363 | 0.00598 | 3.364 | 3.109 |
| W3152-r11.135.5-zAb17-m16 | 0.000632 | 3.226 | 0.004718 | 2.211 | 0.01028 | 3.379 | 3.0769 |
| W3152-r11.135.5-zAb17-m17 | 0.001082 | 3.247 | 0.007182 | 2.345 | 0.01164 | 3.412 | 3.1586 |

TABLE 5
Koff to human PD-Ll by SPR

| Antibody | Koff (1/s) | R0 (RU) |
|---|---|---|
| W315-BMK1-uIgG4K | 1.22E−03 | 165.78 |
| W3152-r11.135.5-zAb17-IgG4L | Weak binding | |
| W3152-r11.135.5-zAb17-m1-uIgG4L | 7.15E−03 | 98.13 |
| W3152-r11.135.5-zAb17-m2-uIgG4L | 1.17E−02 | 63.2 |
| W3152-r11.135.5-zAb17-m3-uIgG4L | Low expression | |
| W3152-r11.135.5-zAb17-m4-uIgG4L | 6.49E−03 | 97.17 |
| W3152-r11.135.5-zAb17-m5-uIgG4L | 2.64E−03 | 70.13 |
| W3152-r11.135.5-zAb17-m6-uIgG4L | 1.03E−03 | 99.36 |
| W3152-r11.135.5-zAb17-m7-uIgG4L | 3.22E−03 | 63.88 |
| W3152-r11.135.5-zAb17-m8-uIgG4L | 1.16E−02 | 69.36 |
| W3152-r11.135.5-zAb17-m9-uIgG4L | 6.79E−03 | 49.16 |
| W3152-r11.135.5-zAb17-m10-uIgG4L | 6.28E−03 | 70.56 |
| W3152-r11.135.5-zAb17-m11-uIgG4L | 6.16E−03 | 46.81 |
| W3152-r11.135.5-zAb17-m12-uIgG4L | 5.55E−03 | 119.34 |
| W3152-r11.135.5-zAb17-m13-uIgG4L | 3.55E−03 | 131.9 |
| W3152-r11.135.5-zAb17-m14-uIgG4L | 6.86E−03 | 66.74 |
| W3152-r11.135.5-zAb17-m15-uIgG4L | 1.41E−03 | 96.43 |
| W3152-r11.135.5-zAb17-m16-uIgG4L | Low expression | |
| W3152-r11.135.5-zAb17-m17-uIgG4L | 1.42E−03 | 87.42 |

### Example 4: Antibody characterization

1. Affinity to human and cynomolgus PD-L1by SPR

Antibodies were characterized for affinity and binding kinetics to PD-L1by SPR assay using ProteOn XPR36 (Bio-Rad). Protein A protein (Sigma) was immobilized to a GLM sensor chip (Bio-Rad) through amine coupling. Purified antibodies were flowed over the sensor chip and captured by the Protein A. The chip was rotated 90° and washed with running buffer (1 ><PBS/0.05% Tween20, Bio-Rad) until the baseline was stable. Five concentrations of human or cynomolgus monkey PD-L1and running buffer were flowed through the sensor chip at a flow rate of 100 μL/min for an association phase of 240s, followed by 600s dissociation. After regeneration, five concentration of mouse PD-L 1 and running buffer were flowed through the sensor chip at a flow rate of 100 μL/min for an association phase of 240s, followed by 600s dissociation. The chip was regenerated with pH 1.5 H₃PO₄ after each run. The association and dissociation curve was fit by 1:1 Langmuir binding model using ProteOn software.

Table 6 shows the results of full kinetic binding affinity to human PD-L1by SPR. Table 7 shows the results of full kinetic binding affinity to cynomolgus monkey PD-L1 by SPR.

TABLE 6
Affinity to human PD-L1 by SPR

| Analyte | Ligand | ka (1/Ms) | kd (1/s) | KD (M) |
|---|---|---|---|---|
| Human | W3152-r11.135.5- | 2.81E+06 | 1.45E−03 | 5.14E−10 |
| PD-Ll | zAb17-m6-uIgG4L | | | |
| | WBP315BMK1 | 3.74E+06 | 1.11E−03 | 2.97E−10 |
| | WBP315BMK6 | 2.00E+06 | 1.64E−04 | 8.18E−11 |

TABLE 7
Affinity to cynomolgus monkey PD-Ll by SPR

| Analyte | Ligand | ka (1/Ms) | kd (1/s) | KD (M) |
|---|---|---|---|---|
| Cynomolgus | W3152-r11.135.5- | 2.52E+06 | 1.01E−03 | 4.03E−10 |
| monkey | zAb17-m6-uIgG4L | | | |
| PD-Ll | WBP315BMK1 | 1.10E+07 | 3.13E−02 | 2.84E−09 |
| | WBP315BMK6 | 1.26E+06 | 1.26E−04 | 1.00E−10 |

2. Cross-reactivity to human, cynomolgus and mouse PD-L1(cross-species)

Cross-reactivity was measured by FACS. Briefly, constructed cell lines that respectively express human, cynomolgus or mouse PD-L 1 were transferred in to 96-well U-bottom plates (BD) at a density of 2×10⁵ cells/well. Testing antibodies were diluted in wash buffer (1><PBS/1% BSA) and incubated with cells at 4° C. for 1 hour. After washing, the secondary antibody goat anti-human IgG Fc FITC (Jackson ImmunoResearch Lab) was added and incubated at 4° C. in the dark for 1 hour. The cells were then washed once and resuspended in 1><PBS/1% BSA, and analyzed by flow cytometry (BD).

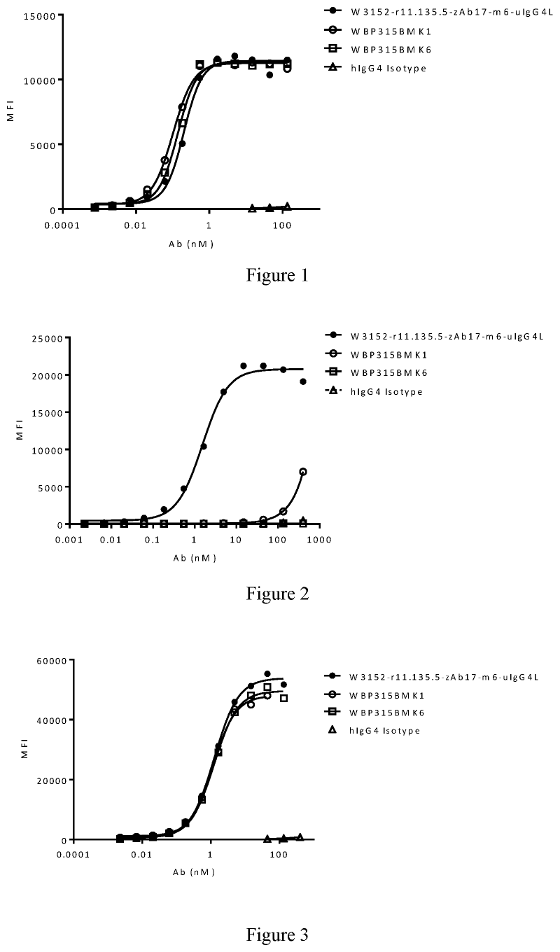

The data for binding of anti-PD-L1 antibodies to cell surface human, mouse and cynomolgus PD-L1is shown in FIG. 1, FIG. 2 and FIG. 3, respectively. The data shows that the antibody W3 152-r11.135.5-zAb17-m6-uIgG4L binds to cell surface human, mouse and cynomolgus PD-L1in a dose-dependent manner.

3. Cross-reactivity to human PD-L1family member, PD-L2 (cross-families)

Constructed cell lines that respectively express human PD-L1 or PD-L2 were transferred in to 96-well U-bottom plates (BD) at a density of 2X 10⁵ cells/well. Testing antibodies were diluted in wash buffer (1><PBS/1% BSA) and incubated with cells at 4° C. for 1 hour. After washing, the secondary antibody goat anti-human IgG Fc FITC (Jackson ImmunoResearch Lab) was added and incubated at 4° C. in the dark for 1 hour. The cells were then washed once and resuspended in 1><PBS/1% BSA, and analyzed by flow cytometry (BD).

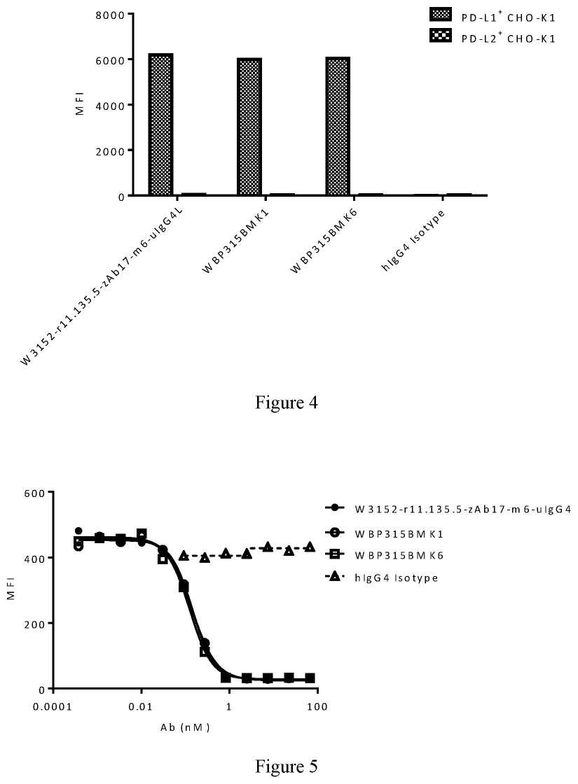

Result of cross family binding test of anti-PD-L1 antibody to human PD-L2 is shown in FIG. 4. The result demonstrates that the antibody W3152-r11.135.5-zAb17-m6-uIgG4L specifically binds to PD-L1, and does not to PD-L2.

4. Blocking of ligands binding to PD-L1

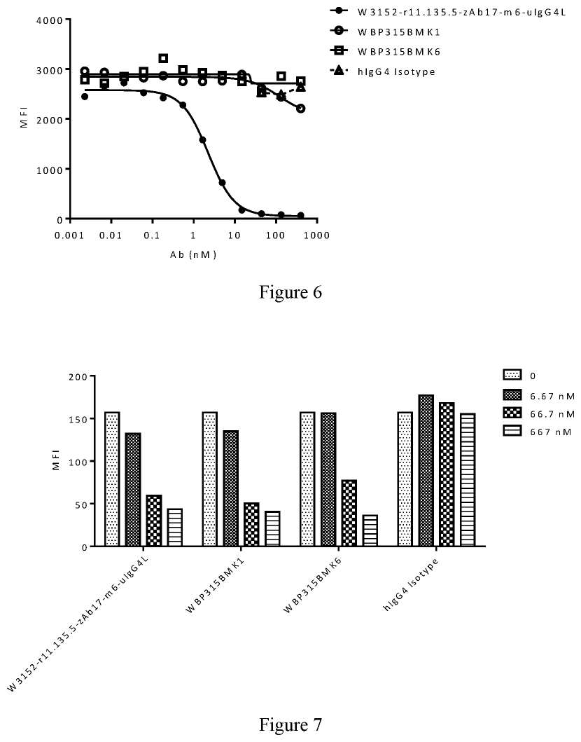

The ability of anti-PD-L1antibodies to block PD-1 binding to PD-L1was tested by FACS as described in Embodiment 3.1.2. The result is shown in FIG. 5 and FIG. 6. The data demonstrates that the antibody W3152-r11.135.5-zAb17-m6-uIgG4L can block human PD-1 binding to PD-L1, and mouse PD-1 binding to PD-L 1 in a dose-dependent manner.

The ability of anti-PD-L1antibodies to block CD80 binding to PD-L1was tested by FACS. Briefly, the anti-PD-L1antibodies and human Fc fusion-PD-L1protein were pre-incubated for 1 hour at 4° C., and then were transferred to 96-well U-bottom plate with 2X 10⁵ cells/well CD80 transfectant CHO-K1 cells. After 1-hour incubation at 4° C. cells were washed and incubated with goat anti-human IgG Fc (Jackson ImmunoResearch Lab) to detect the binding of PD-L1to CD80. After incubation at 4° C. in the dark for 1 hour, the cells were washed once and resuspended in 1><PBS/1% BSA, and analyzed by flow cytometry (BD).

The result of PD-L1/CD80 binding competition is shown in FIG. 7. The data demonstrates that the antibody W3152-r11.135.5-zAb17-m6-uIgG4L can block human CD80 binding to PD-L1in a dose-dependent manner.

5. In vitro function of anti-PD-L1antibodies tested by cell-based assays 5.1 Human allogeneic mixed lymphocyte reaction (MLR)

Human PBMCs were freshly isolated from healthy donors using Ficoll-Paque PLUS (GE) gradient centrifugation. Monocytes were isolated using Human Monocyte Enrichment Kit (StemCell) according to the manufacturer's instructions. Cells were cultured in medium containing recombinant human GM-CSF and IL-4 for 5 to 7 days to differentiate into dendritic cells. 18 to 24 hours before MLR, 1 μg/mL LPS was added to the culture to induce the maturation of the DCs. Human CD4⁺ T cells were isolated using Human CD4+T Cell Enrichment Kit (StemCell) according to the manufacturer's protocol.

Mixed Lymphocyte Reaction was used to test the effects of anti-PD-L1antibodies on modulating T lymphocytes function. Briefly, primary dendritic cell (DC)-stimulated MLR was conducted in 96-well, U-bottom tissue culture plates in 200 μL of RPMI 1640 containing 10% FCS and 1% antibiotics. DCs were mixed with 1×10⁵ CD4⁺ T cells at a DC:T cells ratio between 1:10 and 1:200, in the presence or absence of testing antibodies or benchmark antibodies (form 166.75 nM down to 0.00667 nM, generally total six concentrations). To determine the effect of anti-PD-L1 antibodies on T cell function, the cytokine production and T cell proliferation were determined.

The CD4+T cells proliferation was determined by 3H-thymidine incorporation assay. 3H-thymidine (cat# PerkinElmer-NET027001MC) was diluted 1:20 in 0.9% NaCl solution, and added to the cell culture plates at 0.5 μCi/well. The plates were cultured in 5% CO₂ at 37° C. for 16 to 18 hours, before the incorporation of 3H-thymidine into the proliferating cells was determined.

Human IFN-γ and IL-2 were measured by ELISA using matched antibody pairs. The plates were pre-coated with capture antibody specific for human IFN-γ (cat# Pierce-M700A) or IL-2 (cat# R&D-MAB602), respectively. The biotin-conjugated anti-IFN-γ antibody (cat# Pierce-M701B) or anti-IL-2 antibody (cat# R&D-BAF202) was used as detecting antibody.

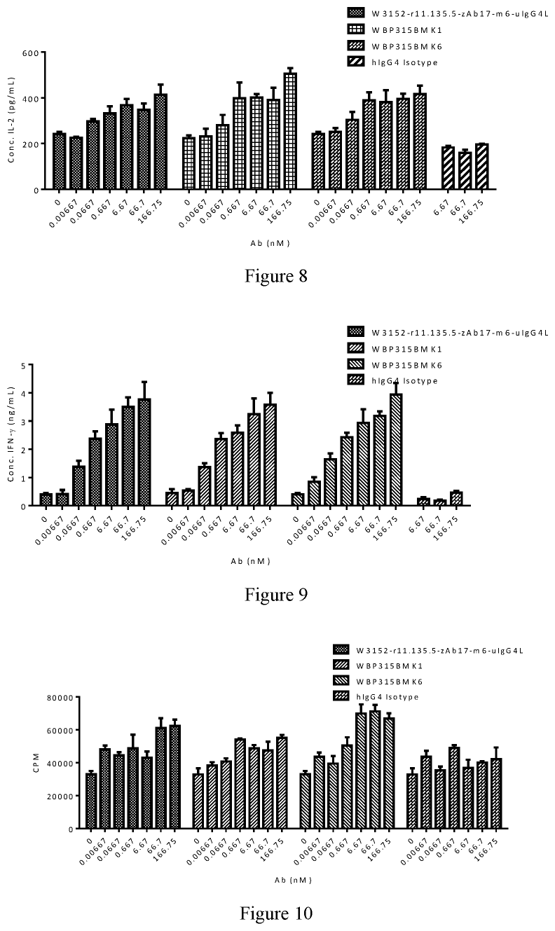

FIGS. 8, 9 and 10 show the effect of anti-PD-L1 antibodies on IL-2 and IFN-γ secretion and T cell proliferation, respectively. It is demonstrated that the antibody W3152-r11.135.5-zAb17-m6-uIgG4L can enhance human IL-2 and IFN-γ secretion and promote T cell proliferation. 5.2 Human autologous mixed lymphocyte reaction

In this assay, the CD4⁺ T cells and DCs were from a same donor. Briefly, freshly isolated PBMC were cultured in the presence of CMV pp65 peptide and low dose of IL-2 (generally 20 U/mL). At the meanwhile, DCs were generated by culturing monocytes from the same donor's PBMC in recombinant human GM-CSF and IL-4. After 5 days, CD4+T cells enriched from the CMV pp65 peptide treated PBMC were co-cultured with DCs which were pre-pulsed with the same peptide for 1 hour in the absence or presence of anti-PD-L1antibodies or benchmark antibodies (as control). On day 5, 100 μL of supernatants were taken from each of cultures for IFN-γ measurement by ELISA as described above. The proliferation of CMV pp65-specific T cells was assessed by 3H-thymidine incorporation as described above.

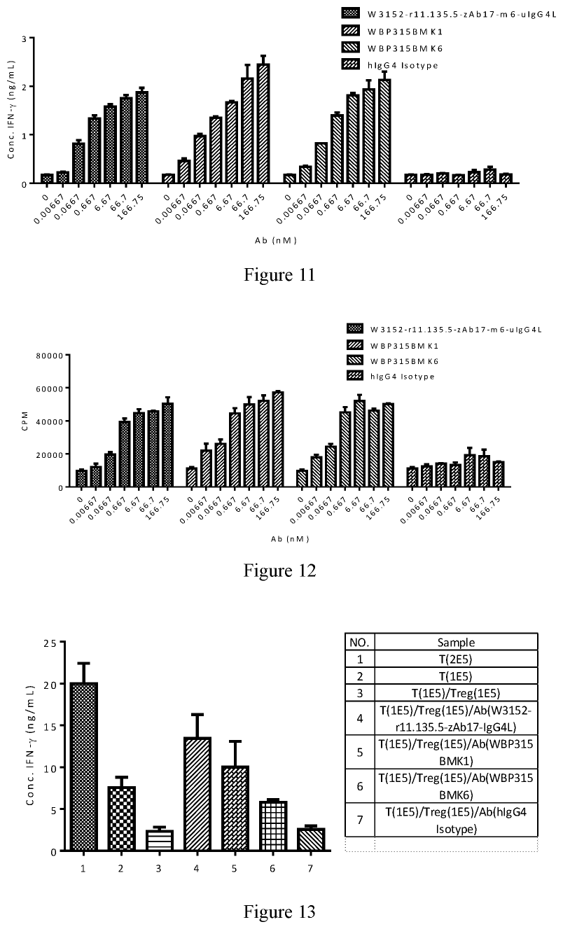

FIGS. 11 and 12 show the results of human auto-MLR demonstrating the anti-PD-L1 antibodies can enhance the function of human CD4+T cell. FIG. 11 shows W3152-r11.135.5-zAb17-m6-uIgG4L increases IFN-γ secretion in a dose-dependent manner. FIG. 12 shows W3152-01.135.5-zAb17-m6-u1gG4L increases CD4+T cells proliferation in a dose-dependent manner.

5.3 Effect of anti-PD-L1antibodies on regulatory T cells (Tregs) suppressive function

Tregs, a subpopulation of T cells, are a key immune modulator and play critical roles in maintaining self-tolerance. Increased numbers of CD4⁺CD25⁺ Tregs were found in patients with multiple cancers and associated with poor prognosis. To determine whether the anti-PD-L1antibodies affect the immune suppressive role of Tregs, we compared the T cell function in the presence of Tregs with or without anti-PD-L 1 antibody treatment. CD4⁺CD25⁺and CD4⁺CD25⁻ T cells were separated using specific anti-CD25 microbeads (StemCell) per manufacture's instruction. Two thousand mature DCs, 1 ×10⁵ CD4±CD25⁻ T cells, 1 ×10⁵ Treg cells and anti-PD-L 1 antibodies were incubated in 96-well plates. The plates were kept at 37° C. in a 5% CO₂ incubator for 5 days. IFN-γ production and CD4⁺CD25⁻ T cells proliferation were determined as described above.

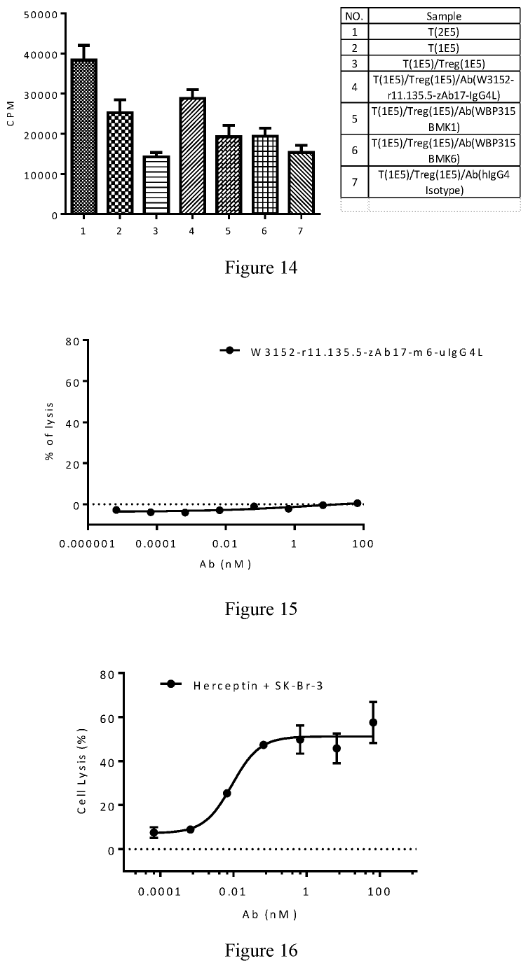

FIGS. 13 and 14 show the effect of antibodies on Treg cells. The antibody W3152-r11.135.5-zAb17-uIgG4L can restore CD4±CD25⁻ T cells secreting IFN-γ (FIG. 13) and proliferation (FIG. 14) by reversing the suppressive function of Treg cells.

6. ADCC and CDC test

PD-L1is expressed on variety of cell types. In order to minimize the potential toxicity to healthy PD-L1positive cells, the anti-PD-L1antibodies were evaluated for their ability to mediate antibody-dependent cell-mediated cytotoxicity (ADCC) and complement-dependent cytotoxicity (CDC).

6.1 ADCC test

Human dendritic cells and various concentrations of anti-PD-L 1 antibodies were pre-incubated in 96-well plate for 30 minutes, and then PBMCs were added at the effector/target ratio of 50:1. The plate was kept at 37° C. in a 5% CO₂ incubator for 6 hours. Target cell lysis was determined by LDH-based cytotoxicity detection kit (cat# Roche-11644793001). The absorbance at 492 nm was read using a microplate reader (Molecular Device). Herceptin-induced SK-Br-3 cell lysis was used as positive control.

FIGS. 15 and 16 show the ADCC effect of anti-PD-L1 antibodies on PD-L1 positive dendritic cells (FIG. 15) and Herceptin on SK-Br-3 (FIG. 16), demonstrating the antibody W3152-r11. 135.5 -zAb 17-m6-uIg G4L didn't mediate ADCC activity on dendritic cells.

6.2 CDC test

Human dendritic cells and various concentrations of anti-PD-L 1 antibodies were mixed in 96-well plate. Human complement was added at the dilution ratio of 1:50. The plate was kept at 37° C. in a 5% CO₂ incubator for 2 hours. Target cell lysis was determined by CellTiter-Glo. Rituxan®-induced Ramos cell lysis was used as positive control. The luminescence was read using a microplate reader (Molecular Device).

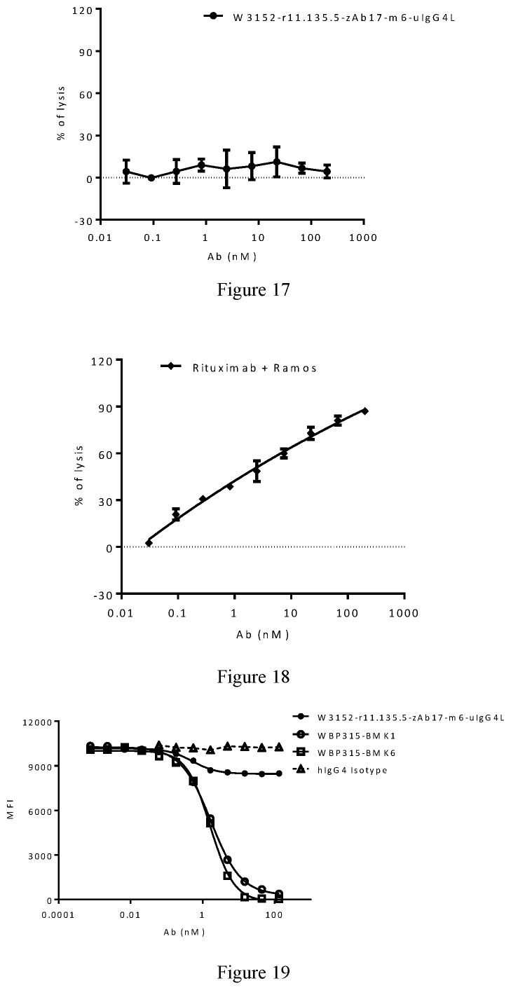

FIGS. 17 and 18 show the CDC effect of anti-PD-L 1 antibodies on PD-L 1 positive dendritic cells (FIG. 17) and Rituximab on Ramos (FIG. 18), demonstrating the antibody W3152-r11.135.5-zAb17-m6-uIgG4L didn't mediate CDC activity on dendritic cells. 7. Epitope binning test

The binding epitope of anti-PD-L 1 antibodies was compared with benchmark antibodies by FACS. CHO-K 1 cells expressing human PD-L1on the cell surface were incubated with the mixture of biotinylated benchmark antibodies and testing antibodies (serially diluted in wash buffer) at 4° C. for 1 hour. The cells were washed and the second antibody Streptavidin-PE were added and incubated for 30 min at 4° C. The cells were then washed once and resuspended in 1><PBS/1% BSA, and analyzed by flow cytometery.

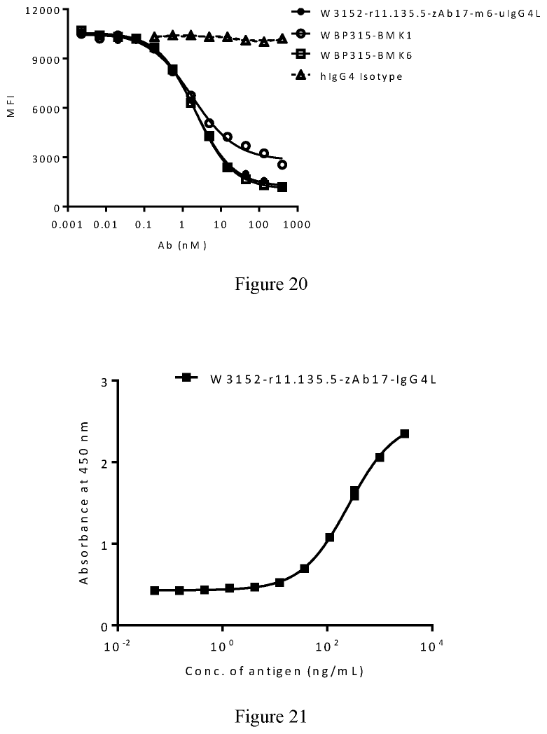

FIGS. 19 and 20 show the results of epitope binning of the antibody W3152-r11. 135.5-zAb17-m6-uIgG4L against WBP315BMK1 (FIG. 19) and WBP315BMK6 (FIG. 20). As shown in the figures, W3152-r11.135.5-zAb17-m6-uIgG4L share different bin from WBP315BMK1, but similar or close bin with WBP315BMK6.

8. Epitope mapping

Alanine scanning experiments on human PD-L 1 were conducted and their effect to antibody binding was evaluated Alanine residues on human PD-L1were mutated to glycine codons, and all other residues were mutated to alanine codons. For each residue of the human PD-L1 ECD, point amino acid substitutions were made using two sequential PCR steps. A pcDNA3.3-hPD-L1 _ECD. His plasmid that encodes ECD of human PD-L1and a C-terminal His-tag was used as template, and a set of mutagenic primer was used for first step PCR using the QuikChange lightning multi site-directed mutagenesis kit (Agilent technologies, Palo Alto, Calif.). Dpn I endonuclease was used to digest the parental template after mutant strand synthesis reaction. In the second-step PCR, linear DNA expression cassette which composed of a CMV promoter, an ECD of PD-L1, a His-tag and a herpes simplex virus thymidine kinase (TK) polyadenylation was amplified and transiently expressed in HEK293F cells (Life Technologies, Gaithersburg, Md.).

Monoclonal antibodies W3152-r11.135.5-zAb17-IgG4L (2 μg/mL) was add to plates which pre-coated with 2 μg/mL Goat-anti-human-IgG Fc (Bethyl Laboratories, Montgomery, TX) in plates for ELISA binding assay. After interacting with the supernatant that contains quantified PD-L1mutant or human PD-L1-ECD-His protein (Sino Biological, China), HRP conjugated anti-His antibody (1:5000; Rockland Immunochemicals, Pottstown, PA) was added as detection antibody. Absorbance was normalized according to the average of control mutants. After setting an additional cutoff to the binding fold change (<0.55), the final determined epitope residues were identified.

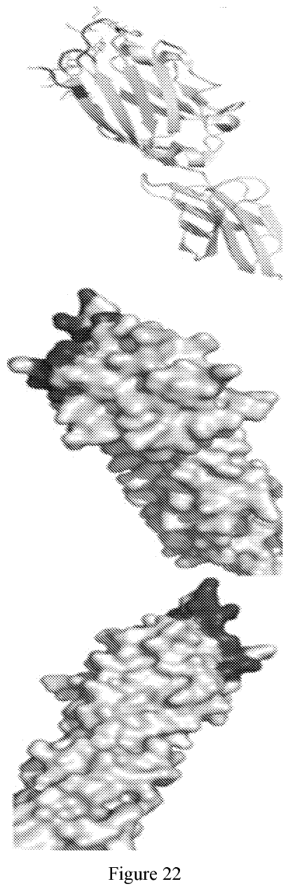

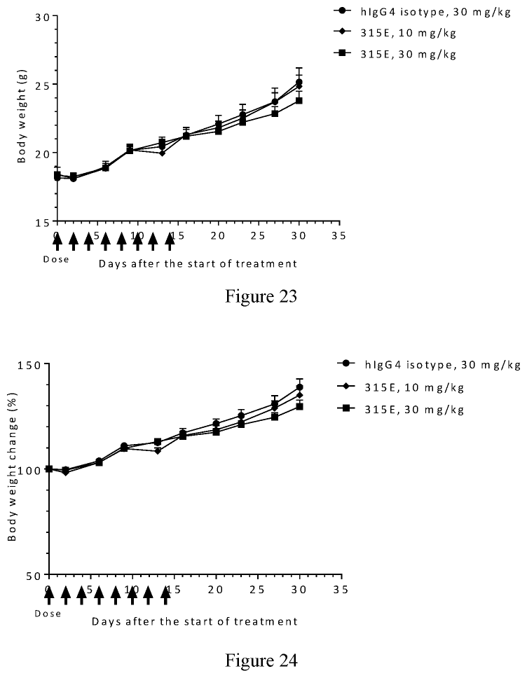

The binding activity of the antibody W3152-r11.135.5-zAb17-IgG4L to human PD-L1was conducted, and the result is shown in FIG. 22.

The effect of 131 PD-L1point mutations on antibody binding is shown in Table 8. Checking the positions of all these residues on the hPD-L1 crystal structures (PDB code 3BIK and 4ZQK) revealed that some amino acids (e.g. Tyr160, Thr201) were unlikely to directly contact any antibodies. The observed binding reductions most probably resulted from the instability or even collapse of hPD-L1 structure after alanine substitutions. After setting an additional cutoff to the binding fold change (<0.55), the final determined epitope residues are listed in Table 9. They are 15 residues to W3152-r11.135.5-zAb17-IgG4L.

All data in Table 9 are therefore mapped on the crystal structure of hPD-L1 to make a better visualization and comparison (FIG. 16). Colors on the pictures are to help distinguish the differences between epitopes.

As shown in FIG. 16, the hot-spot residues in charge of the human PD-L1 binding all gathered in the A strand, BC loop, C′D loop, F strand, FG loop and G strands. The investigated antibody W3152-r11.135.5-zAb17-IgG4L is functional in binding PD-LL and blocking PD-1, have almost the same epitopes. Checking the positions of the residues on the hPD-1/hPD-L1 complex crystal structures (PDB code 4ZQK, 4A) revealed that these residues mainly located on A, C, F and G strands. The epitopes of W3152-r11.135.5-zAb17-IgG4L were mainly contributed by the residues on the BC loop, which didn't intersect the PD-L1 binding site at all. This suggested the PD-1 blocking function of W3152-r11.135.5-zAb17-IgG4L relied more on their steric hindrance effects provided by the size of the antibody.

TABLE 8
Effect of PD-Ll point mutations on antibody binding
W3152-r11.135.5-zAb17-IgG4L

| | | fold | |
|---|---|---|---|
| PD-Ll | # Residue | change ^{a} | SD |
| F | 19 | 0.311 | 0.001 |
| T | 20 | 0.325 | 0.001 |
| V | 21 | 0.349 | 0.006 |
| E | 45 | 0.349 | 0.007 |
| D | 122 | 0.372 | 0.008 |
| K | 46 | 0.372 | 0.008 |
| L | 48 | 0.385 | 0.026 |
| T | 22 | 0.399 | 0.010 |
| T | 201 | 0.424 | 0.001 |
| G | 70 | 0.430 | 0.006 |
| D | 49 | 0.445 | 0.019 |
| V | 44 | 0.464 | 0.061 |
| P | 43 | 0.472 | 0.067 |
| Y | 118 | 0.497 | 0.014 |
| G | 119 | 0.502 | 0.018 |
| G | 120 | 0.521 | 0.021 |
| Y | 160 | 0.531 | 0.018 |
| A | 52 | 0.564 | 0.018 |
| N | 131 | 0.570 | 0.022 |
| P | 216 | 0.573 | 0.017 |
| V | 68 | 0.582 | 0.027 |
| Q | 66 | 0.596 | 0.001 |
| L | 50 | 0.617 | 0.051 |
| G | 110 | 0.623 | 0.033 |
| P | 133 | 0.624 | 0.006 |
| Y | 32 | 0.628 | 0.078 |
| L | 106 | 0.644 | 0.090 |
| Q | 47 | 0.679 | 0.005 |
| E | 72 | 0.686 | 0.006 |
| G | 33 | 0.705 | 0.047 |
| T | 37 | 0.721 | 0.009 |
| E | 71 | 0.724 | 0.014 |
| L | 88 | 0.729 | 0.006 |
| K | 129 | 0.735 | 0.027 |
| P | 161 | 0.735 | 0.014 |
| K | 41 | 0.737 | 0.002 |
| V | 128 | 0.739 | 0.039 |
| V | 111 | 0.744 | 0.041 |
| R | 113 | 0.745 | 0.039 |
| G | 95 | 0.747 | 0.003 |
| G | 159 | 0.753 | 0.003 |
| D | 26 | 0.756 | 0.006 |
| A | 121 | 0.760 | 0.013 |
| D | 103 | 0.760 | 0.006 |
| E | 60 | 0.762 | 0.004 |
| V | 23 | 0.769 | 0.015 |
| K | 105 | 0.776 | 0.015 |
| T | 196 | 0.778 | 0.043 |
| E | 218 | 0.779 | 0.022 |
| P | 24 | 0.779 | 0.033 |
| T | 102 | 0.787 | 0.014 |
| E | 58 | 0.790 | 0.003 |
| A | 18 | 0.791 | 0.037 |
| K | 89 | 0.791 | 0.018 |
| D | 215 | 0.792 | 0.028 |
| D | 90 | 0.794 | 0.042 |
| E | 217 | 0.795 | 0.008 |
| V | 225 | 0.810 | 0.043 |
| Y | 56 | 0.811 | 0.007 |
| T | 127 | 0.816 | 0.046 |
| S | 34 | 0.819 | 0.033 |
| Y | 81 | 0.823 | 0.050 |
| L | 142 | 0.828 | 0.015 |
| R | 86 | 0.829 | 0.066 |
| K | 25 | 0.835 | 0.112 |
| I | 64 | 0.840 | 0.012 |
| A | 98 | 0.843 | 0.087 |
| D | 61 | 0.845 | 0.006 |
| E | 237 | 0.845 | 0.005 |
| I | 38 | 0.851 | 0.015 |
| R | 82 | 0.856 | 0.030 |
| K | 62 | 0.856 | 0.025 |
| N | 63 | 0.857 | 0.010 |
| A | 109 | 0.858 | 0.109 |
| Y | 28 | 0.863 | 0.007 |
| Q | 107 | 0.866 | 0.062 |
| Q | 100 | 0.869 | 0.066 |
| Y | 123 | 0.874 | 0.026 |
| N | 96 | 0.874 | 0.048 |
| T | 180 | 0.874 | 0.011 |
| F | 191 | 0.874 | 0.037 |
| N | 35 | 0.878 | 0.001 |
| E | 31 | 0.893 | 0.028 |
| S | 79 | 0.896 | 0.002 |
| H | 78 | 0.897 | 0.093 |
| N | 135 | 0.900 | 0.032 |
| S | 93 | 0.902 | 0.030 |
| L | 231 | 0.906 | 0.123 |
| S | 117 | 0.906 | 0.026 |
| E | 205 | 0.908 | 0.029 |
| V | 143 | 0.920 | 0.027 |
| A | 232 | 0.922 | 0.125 |
| T | 179 | 0.923 | 0.016 |
| I | 54 | 0.924 | 0.000 |
| V | 29 | 0.925 | 0.037 |
| R | 186 | 0.928 | 0.025 |
| L | 214 | 0.929 | 0.068 |
| Q | 91 | 0.932 | 0.008 |
| Q | 83 | 0.935 | 0.028 |
| E | 188 | 0.936 | 0.015 |
| N | 236 | 0.938 | 0.072 |
| I | 206 | 0.941 | 0.003 |
| M | 59 | 0.941 | 0.011 |
| M | 36 | 0.941 | 0.012 |
| A | 51 | 0.944 | 0.010 |
| L | 94 | 0.946 | 0.032 |
| K | 185 | 0.946 | 0.014 |
| Q | 77 | 0.953 | 0.037 |
| S | 80 | 0.953 | 0.054 |
| T | 154 | 0.960 | 0.014 |
| M | 115 | 0.964 | 0.037 |
| E | 39 | 0.965 | 0.014 |
| K | 189 | 0.965 | 0.011 |
| H | 69 | 0.968 | 0.000 |
| R | 125 | 0.969 | 0.016 |
| T | 181 | 0.971 | 0.004 |
| V | 76 | 0.973 | 0.046 |
| D | 73 | 0.985 | 0.018 |
| N | 183 | 0.987 | 0.002 |
| T | 203 | 0.992 | 0.048 |
| I | 199 | 0.998 | 0.035 |
| L | 27 | 1.007 | 0.015 |
| V | 147 | 1.012 | 0.018 |
| K | 75 | 1.012 | 0.007 |
| S | 169 | 1.046 | 0.002 |
| L | 74 | 1.058 | 0.087 |
| K | 162 | 1.077 | 0.026 |
| K | 124 | 1.089 | 0.002 |
| V | 174 | 1.110 | 0.025 |
| L | 175 | 1.126 | 0.044 |
| R | 212 | 1.927 | 0.030 |

^{a} Fold change in binding is relative to the binding of several silent alanine substitutions.

TABLE 9
Effect of PD-L1 point mutations on antibody binding

| PD-L1 to W3152- | |
|---|---|
| r11.135.5-zAb17- | |
| IgG4L | residue location |
| F 19 | Ahead of A strand |
| T 20 | Ahead of A strand |
| V 21 | A strand |
| T 22 | A strand |
| P 43 | BC loop |
| V 44 | BC loop |
| E 45 | BC loop |
| K 46 | BC loop |
| L 48 | BC loop |
| D 49 | BC loop |
| G 70 | C’D loop |
| Y 118 | F strand |
| G 119 | FG loop |
| G 120 | G strand |
| D 122 | G strand |

9. In vivo efficacy study 9.1 Experiment design

TABLE 10
Grouping and dosing regimen of the in vivo animal efficacy experiments

| | | | | Dose-Volume | | |
|---|---|---|---|---|---|---|
| | | | Dose | Parameters | Route of | Frequency of |
| group | N¹ | Treatment | (mg/kg) | (μL/g)² | administration | administration |
| 1 | 7 | hIgG4 Isotype | 30 mg/kg | 10 | IP | Q2D × 8 |
| 2 | 7 | W3152-r11.135.5- | 10 mg/kg | 10 | IP | Q2D × 8 |
| | | zAb17-m6-IgG4L | | | | |
| 3 | 7 | W3152-r11.135.5- | 30 mg/kg | 10 | IP | Q2D × 8 |
| | | zAb17-m6-IgG4L | | | | |

Note:
¹N: mice number in each group
²Dose-Volume: 10 μL/g according to the weight of mouse. If the weight loss exceeds 15%, the dosing regimen should be adjusted accordingly.

9.2 Methods

9.2.1 Cell Culture

The MC38-huPD-L1 cells, in which human PD-L 1 gene was knocked in, were maintained in vitro as a monolayer culture in 1640 medium supplemented with 10% fetal bovine serum, 1% Hygromycin B at 37° C. in an atmosphere of 5% CO₂ in air. The tumor cells were routinely subcultured twice weekly by trypsin-EDTA treatment. The cells growing in an exponential growth phase were harvested and counted for tumor inoculation.

9.2.2 Tumor inoculation

Each mouse was inoculated subcutaneously at the right flank with MC38-huPD-L1 tumor cells (5 ×10⁵) in 0.1 mL PBS. The treatments were started on when the average tumor size reached approximately 144 mm³ The testing article was administrated to the mice according to the predetermined regimen as shown in the experimental design table (Table 10).

9.2.3 Tumor measurements and endpoints

The major endpoint was to see if the tumor growth could be delayed or mice could be cured. Tumor size was measured 2 or 3 per week in two dimensions using a caliper, and the volume was expressed in mm³ using the formula: V=0.5 a X b² where a and b are the long and short diameters of the tumor, respectively. TGI was calculated for each group using the formula: TGI (%)=[1-(Tᵢ-Tₒ)/(Vᵢ-Vₒ)] ×100; Tᵢ was the average tumor volume of a treatment group on a given day, To was the average tumor volume of the treatment group on the day of treatment started, Vᵢ was the average tumor volume of the vehicle control group on the same day as Tᵢ, and Vₒ was the average tumor volume of the vehicle group on the day of treatment started.

9.3 Results

9.3.1 Animal observation and body weight change

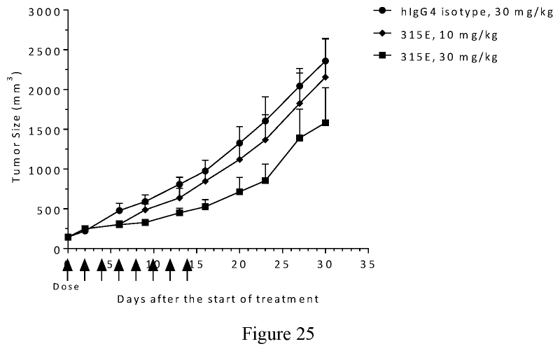

No obvious clinical signs were observed during the entire study. Body weights of all animals gradually increased during the study. The results of body weight and body weight change are shown in FIGS. 24 and 25. No obvious clinical signs were observed during the study. Therefore, no obvious toxicity was observed in MC38-huPD-L1 model.

9.3.2 Tumor growth inhibition

All mice were closely monitored for tumor growth during the entire experiment, with tumor size measured and recorded twice a week. The tumor growth inhibition (TGI_{TV}) was calculated and analyzed at the best therapeutic time-point (16 days post grouping). The results of tumor volume were shown in FIG. 26 and summarized in Table 11 and Table 12.

TABLE 11
Summary of tumor volume

| | Tumor Volume (mm³) ^{a} | | |
|---|---|---|---|
| Days post | hIgG4 | 315E | 315E |
| grouping | Isotype | 10 mg/kg | 30 mg/kg |
| 0 | 144 ± 8 | 144 ± 11 | 144 ± 11 |
| 2 | 221 ± 26 | 247 ± 28 | 251 ± 20 |
| 6 | 478 ± 91 | 303 ± 44 | 299 ± 30 |
| 9 | 590 ± 84 | 487 ± 72 | 32833± |
| 13 | 808 ± 89 | 637 ± 119 | 449 ± 57 |
| 16 | 975 ± 135 | 847 ± 134 | 526 ± 89 |
| 20 | 1,326 ± 207 | 1,121 ± 201 | 714 ± 181 |
| 23 | 1,604 ± 305 | 1,367 ± 315 | 853 ± 208 |
| 27 | 2,044 ± 220 | 1,825 ± 382 | 1,391 ± 362 |
| 30 | 2,358 ± 276 | 2,155 ± 486 | 1,583 ± 441 |

Note:
^{a} Mean ± SEM

TABLE 1
Tumor growth inhibition

| | | Tumor Volume (mm³) ^{a} | | | |
|---|---|---|---|---|---|
| | | Before | 16 days post | | |
| Group | Test articles | grouping | grouping | TGI _{TV} (%) | P value ^{b} |
| G1 | hIgG1 | 144 ± 6 | 975 ± 135 | — | — |
| G2 | 315E, 10 mg/kg | 144 ± 6 | 847 ± 134 | 15.5 | 0.512 |
| G3 | 315E, 30 mg/kg | 144 ± 5 | 526 ± 89 | 54.1 | 0.017 |

Note:
^{a} Mean ± SEM
^{b} p value is calculated based on tumor size using T-test, treatment groups vs isotype control group.

9.3.3 Conclusion

In this study, we have evaluated the in vivo efficacy of 315E, dosed at 10 mg/kg and 30 mg/kg, in the treatment of MC38-huPD-L1 murine colon carcinoma that had engrafted into PD-1 humanized mice. During this study, the body weights of all animals were gradually increased. The body weights of all treated animals had no obviously difference to the hIgG4 isotype control group, indicating the absence of toxicity of the test article in all tested dose levels. On day 16 after administration, the mean tumor volumes of 315E-treated groups, 315E 10 mg/kg and 315E 30 mg/kg, are 847±134 mm³ and 526±89 mm³, respectively. The TGI_{TV} values are 15.5% and 54.1%, respectively. Based on statistics analysis, 315E 30 mg/kg had significant smaller mean tumor volume compared with hIgG4 isotype control group (P<0.05).

The test article 315E exhibited significant inhibition effect on tumor growth at the dose level of 30 mg/kg, while only limited anti-tumor activity at the dose level of 10 mg/kg. All the grouped mice tolerated well to all dose levels tested.

1. An antibody or antigen binding fragment thereof that binds to an epitope of human and murine PD-L1, wherein the epitope comprises amino acids at positions 19-22, 43-46, 48-49, 70, 118-120, 122 of SEQ ID NO:1.

2. The antibody or the antigen binding fragment thereof of claim 1, wherein the murine PD-L1is mouse or rat PD-L1.

3. An antibody, or antigen-binding fragment thereof, comprising:
a heavy chain variable domain comprising CDR1, CDR2, and CDR3 sequences; and a light chain variable domain comprising CDR1, CDR2, and CDR3 sequences,
wherein the heavy chain variable region CDR3 sequence comprises a sequence of SEQ ID NO: 4, and conservative modifications thereof,
the heavy chain variable region CDR2 sequence comprises a sequence of SEQ ID NO: 5, and conservative modifications thereof,
the heavy chain variable region CDR1 sequence comprises a sequence of SEQ ID NO: 6, and conservative modifications thereof,
the light chain variable region CDR3 sequence comprises a sequence of SEQ ID NO: 7, and conservative modifications thereof,
the light chain variable region CDR2 sequence comprises a sequence of SEQ ID NO: 8, and conservative modifications thereof,
the light chain variable region CDR1 sequence comprises a sequence of SEQ ID NO: 9, and conservative modifications thereof,
wherein the antibody specifically binds to PD-L1.

4. An antibody or an antigen binding fragment thereof of claim 3, comprising a heavy chain variable (VH) domain comprising an amino acid sequence of SEQ ID NO: 2 or an amino acid sequence that is at least 70%, 80%, 90% or 95% identity to a sequence of SEQ ID NO: 2,
wherein the antibody specifically binds to PD-L1.

5. The antibody or the antigen binding fragment thereof of claim 3, further comprising a light chain variable (VL) domain comprising an amino acid sequence of SEQ ID No 3 or an amino acid sequence that is at least 70%, 80%, 90% or 95% identity to a sequence of SEQ ID NO: 3.

6. (canceled)

7. (canceled)

8. The antibody or an antigen binding fragment thereof of claim 3, comprising an amino acid sequence of SEQ ID NO:10.

9. A nucleic acid molecule encoding the antibody, or the antigen binding fragment of claim 3.

10. A cloning or expression vector comprising the nucleic acid molecule of claim 9.

11. A host cell comprising one or more cloning or expression vectors of claim 10.

12. A process for the production of an antibody, comprising culturing the host cell of claim 11 and isolating the antibody.

13. (canceled)

14. (canceled)

15. (canceled)

16. A pharmaceutical composition comprising the antibody, or the antigen binding fragment claim 3, and one or more of a pharmaceutically acceptable excipient, diluent or carrier.

17. An immunoconjugate comprising the antibody, or antigen-binding fragment thereof, according to claim 3, linked to a therapeutic agent.

18. A pharmaceutical composition comprising the immunoconjugate of claim 17 and a pharmaceutically acceptable excipient, diluent or carrier.

19. A method for preparing an anti-PD-L1antibody or an antigen-binding fragment thereof comprising:
(a) providing:
(i) a heavy chain variable region antibody sequence comprising a CDR1 sequence of SEQ ID NO: 6, a CDR2 sequence of SEQ ID NO: 5; and a CDR3 sequence of SEQ ID NO: 4; and
(ii) a light chain variable region antibody sequence comprising a CDR1 sequence of SEQ ID NO: 9, a CDR2 sequence of SEQ ID NO: 8, and a CDR3 sequence of SEQ ID NO: 7; and
(b) expressing the antibody sequence as a protein.

20. (canceled)

21. A method for the treatment or prophylaxis of an immune disorder or cancer in the subject comprising administering to the subject a therapeutically effective amount of the antibody, or the antigen-binding fragment of claim 3.

22. (canceled)

23. The method of claim 21, wherein the cancer is selected from a group consisting of melanoma, renal cancer, prostate cancer, breast cancer, colon cancer, lung cancer, bone cancer, pancreatic cancer, skin cancer, cancer of the head or neck, cutaneous or intraocular malignant melanoma, uterine cancer, ovarian cancer, and rectal cancer.

24. The method of claim 21, wherein the antibody is a humanized antibody.

25. A method of the treatment or prophylaxis of an immune disorder or cancer in the subject, comprising administering to the subject a therapeutically effective amount of the antibhody, or the antigen-binding fragment of claim 1.

26. The method of claim 18, wherein the cancer is selected from a group consisting of melanoma, renal cancer, prostate cancer, breast cancer, colon cancer, lung cancer, bone cancer, pancreatic cancer, skin cancer, cancer of the head or neck, cutaneous or intraocular malignant melanoma, uterine cancer, ovarian cancer, and rectal cancer.

27. A nucleic acid molecule encoding the antibody, or the antigen binding fragment of claim 1.
